SEQUENCE LISTING (5 sequences)

SEQ ID NO: 1 - DNA, 1674 bp, synthetic construct
  source 1..1674: mol_type = other DNA
atggaatcagaaagaagcaaaaggatgggaaatgcctgcattcccctgaaaagaattgcttatttcctatgtctcttatctgcgcttttgctgactgaggggaagaaaccagcgaagccaaaatgccctgccgtgtgtacttgtaccaaagataatgctttatgtgagaatgccagatccattccacgcaccgttcctcctgatgttatctcattatcctttgtgagatctggttttactgaaatctcagaagggagttttttattcacgccatcgctgcagctcttgttattcacatcgaactcctttgatgtgatcagtgatgatgcttttattggtcttccacatctagagtatttattcatagaaaacaacaacatcaagtcaatttcaagacatactttccggggactaaagtcattaattcacttgagccttgcaaacaacaatctccagacactcccaaaagatattttcaaaggcctggattctttaacaaatgtggacctgaggggtaattcatttaattgtgactgtaaactgaaatggctagtggaatggcttggccacaccaatgcaactgttgaagacatctactgcgaaggccccccagaatacaagaagcgcaaaatcaatagtctctcctcgaaggatttcgattgcatcattacagaatttgcaaagtctcaagacctgccttatcaatcattgtccatagacactttttcttatttgaatgatgagtatgtagtcatcgctcagccttttactggaaaatgcattttccttgaatgggaccatgtggaaaagaccttccggaattatgacaacattacaggcacatccactgtagtatgcaagcctatagtcattgaaactcagctctatgttattgtggcccagctgtttggtggctctcacatctataagcgagacagttttgcaaataaattcataaaaatccaggatattgaaattctcaaaatccgaaaacccaatgacattgaaacattcaagattgaaaacaactggtactttgttgttgctgacagttcaaaagctggttttactaccatttacaaatggaacggaaacggattctactcccatcaatccttacacgcgtggtacagggacactgatgtggaatatctagaaatagtcagaacacctcagacactcagaacgcctcatttaattctgtctagtagttcccagcgtcctgtaatttatcagtggaacaaagcaacacaattattcactaaccaaactgacattcctaacatggaggatgtgtacgcagtgaagcacttctcagtgaaaggggacgtgtacatttgcttgacaagattcattggtgattccaaagtcatgaaatggggaggctcctcgttccaggatattcagaggatgccatcgcgaggatccatggtgttccagcctcttcaaataaataattaccaatatgcaattcttggaagtgattactcctttactcaagtgtataactgggatgcagagaaagccaaatttgtgaaatttcaggaattaaatgttcaggcaccaagatcattcacacatgtgtccattaataagcgtaattttctttttgcttccagttttaagggaaatacacagatttacaaacatgtcatagttgacttaagcgcatga

SEQ ID NO: 2 - protein, 557 aa, synthetic construct
MESERSKRMGNACIPLKRIAYFLCLLSALLLTEGKKPAKPKCPAVCTCTKDNALCENARSIPRTVPPDVISLSFVRSGFTEISEGSFLFTPSLQLLLFTSNSFDVISDDAFIGLPHLEYLFIENNNIKSISRHTFRGLKSLIHLSLANNNLQTLPKDIFKGLDSLTNVDLRGNSFNCDCKLKWLVEWLGHTNATVEDIYCEGPPEYKKRKINSLSSKDFDCIITEFAKSQDLPYQSLSIDTFSYLNDEYVVIAQPFTGKCIFLEWDHVEKTFRNYDNITGTSTVVCKPIVIETQLYVIVAQLFGGSHIYKRDSFANKFIKIQDIEILKIRKPNDIETFKIENNWYFVVADSSKAGFTTIYKWNGNGFYSHQSLHAWYRDTDVEYLEIVRTPQTLRTPHLILSSSSQRPVIYQWNKATQLFTNQTDIPNMEDVYAVKHFSVKGDVYICLTRFIGDSKVMKWGGSSFQDIQRMPSRGSMVFQPLQINNYQYAILGSDYSFTQVYNWDAEKAKFVKFQELNVQAPRSFTHVSINKRNFLFASSFKGNTQIYKHVIVDLSA

SEQ ID NO: 3 - DNA, 1674 bp, synthetic construct
  source 1..1674: mol_type = other DNA
atggaaagcgagcggagcaagcggatgggcaacgcctgcatccccctgaagcggatcgcctacttcctgtgcctgctgagcgccctgctgctgaccgagggaaagaagcccgccaagcctaagtgccctgccgtgtgcacctgtaccaaggacaacgccctgtgcgagaacgcccggtccatccctagaaccgtgccccccgatgtgatcagcctgagctttgtgcggagcggcttcaccgagatcagcgagggcagcttcctgttcacccccagcctgcagctgctgctgttcaccagcaacagcttcgacgtgatcagcgacgacgccttcatcggcctgccccacctggaatacctgttcatcgagaacaacaacatcaagagcatcagccggcacaccttccggggcctgaagtccctgatccacctgagcctggccaacaacaacctgcagaccctgcccaaggacatcttcaagggcctggacagcctgaccaacgtggacctgagaggcaactccttcaactgcgactgcaagctgaagtggctggtggaatggctgggccacaccaacgccaccgtggaagatatctactgcgagggcccacccgagtacaagaagcggaagatcaacagcctgtccagcaaggacttcgactgcatcatcaccgagttcgccaagagccaggacctgccctaccagagcctgagcatcgacaccttcagctacctgaacgacgagtacgtcgtgatcgcccagccctttaccggcaagtgcatcttcctggaatgggaccacgtggaaaagaccttccggaactacgacaacatcaccggcaccagcaccgtcgtgtgcaagcccatcgtgatcgagacacagctgtacgtgatcgtggctcagctgttcggcggcagccacatctacaagcgggacagcttcgccaacaagttcatcaagatccaggacatcgagatcctgaagatccggaagcccaacgacatcgaaacctttaagattgagaacaactggtacttcgtggtggccgacagcagcaaggccggctttaccaccatctataagtggaacggcaacggcttctacagccaccagtccctgcacgcctggtacagagacaccgacgtggaatatctggaaatcgtgcggaccccccagaccctgagaacccctcacctgatcctgagcagctccagccagcggcccgtgatctaccagtggaacaaggccacccagctgtttaccaaccagaccgacatccccaacatggaagatgtgtacgccgtgaagcacttcagcgtgaagggcgacgtgtacatctgcctgacccggttcatcggcgacagcaaagtgatgaagtggggcggcagcagcttccaggatatccagcggatgcccagccggggcagcatggtgtttcagcccctgcagatcaacaactaccagtacgccatcctgggcagcgactacagcttcacccaggtgtacaattgggacgccgagaaggccaagttcgtgaagttccaggaactgaacgtgcaggcccccagatcctttacccacgtgtcaatcaacaagcggaatttcctgttcgccagctcctttaagggcaacacccagatctataagcacgtgatcgtggacctgagcgcctga

SEQ ID NO: 4 - DNA, 7195 bp, synthetic construct
  source 1..7195: mol_type = other DNA
tcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccagctagcatggaaagcgagcggagcaagcggatgggcaacgcctgcatccccctgaagcggatcgcctacttcctgtgcctgctgagcgccctgctgctgaccgagggaaagaagcccgccaagcctaagtgccctgccgtgtgcacctgtaccaaggacaacgccctgtgcgagaacgcccggtccatccctagaaccgtgccccccgatgtgatcagcctgagctttgtgcggagcggcttcaccgagatcagcgagggcagcttcctgttcacccccagcctgcagctgctgctgttcaccagcaacagcttcgacgtgatcagcgacgacgccttcatcggcctgccccacctggaatacctgttcatcgagaacaacaacatcaagagcatcagccggcacaccttccggggcctgaagtccctgatccacctgagcctggccaacaacaacctgcagaccctgcccaaggacatcttcaagggcctggacagcctgaccaacgtggacctgagaggcaactccttcaactgcgactgcaagctgaagtggctggtggaatggctgggccacaccaacgccaccgtggaagatatctactgcgagggcccacccgagtacaagaagcggaagatcaacagcctgtccagcaaggacttcgactgcatcatcaccgagttcgccaagagccaggacctgccctaccagagcctgagcatcgacaccttcagctacctgaacgacgagtacgtcgtgatcgcccagccctttaccggcaagtgcatcttcctggaatgggaccacgtggaaaagaccttccggaactacgacaacatcaccggcaccagcaccgtcgtgtgcaagcccatcgtgatcgagacacagctgtacgtgatcgtggctcagctgttcggcggcagccacatctacaagcgggacagcttcgccaacaagttcatcaagatccaggacatcgagatcctgaagatccggaagcccaacgacatcgaaacctttaagattgagaacaactggtacttcgtggtggccgacagcagcaaggccggctttaccaccatctataagtggaacggcaacggcttctacagccaccagtccctgcacgcctggtacagagacaccgacgtggaatatctggaaatcgtgcggaccccccagaccctgagaacccctcacctgatcctgagcagctccagccagcggcccgtgatctaccagtggaacaaggccacccagctgtttaccaaccagaccgacatccccaacatggaagatgtgtacgccgtgaagcacttcagcgtgaagggcgacgtgtacatctgcctgacccggttcatcggcgacagcaaagtgatgaagtggggcggcagcagcttccaggatatccagcggatgcccagccggggcagcatggtgtttcagcccctgcagatcaacaactaccagtacgccatcctgggcagcgactacagcttcacccaggtgtacaattgggacgccgagaaggccaagttcgtgaagttccaggaactgaacgtgcaggcccccagatcctttacccacgtgtcaatcaacaagcggaatttcctgttcgccagctcctttaagggcaacacccagatctataagcacgtgatcgtggacctgagcgcctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagatatcagccatggcttcccgccggcggtggcggcgcaggatgatggcacgctgcccatgtcttgtgcccaggagagcgggatggaccgtcaccctgcagcctgtgcttctgctaggatcaatgtgaccggttaacg

SEQ ID NO: 5 - DNA, 7269 bp, synthetic construct
  source 1..7269: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgaattcgagtgcaagtgggttttaggaccaggatgaggcggggtgggggtgcctacctgacgaccgaccccacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattggatccagctagcatggaaagcgagcggagcaagcggatgggcaacgcctgcatccccctgaagcggatcgcctacttcctgtgcctgctgagcgccctgctgctgaccgagggaaagaagcccgccaagcctaagtgccctgccgtgtgcacctgtaccaaggacaacgccctgtgcgagaacgcccggtccatccctagaaccgtgccccccgatgtgatcagcctgagctttgtgcggagcggcttcaccgagatcagcgagggcagcttcctgttcacccccagcctgcagctgctgctgttcaccagcaacagcttcgacgtgatcagcgacgacgccttcatcggcctgccccacctggaatacctgttcatcgagaacaacaacatcaagagcatcagccggcacaccttccggggcctgaagtccctgatccacctgagcctggccaacaacaacctgcagaccctgcccaaggacatcttcaagggcctggacagcctgaccaacgtggacctgagaggcaactccttcaactgcgactgcaagctgaagtggctggtggaatggctgggccacaccaacgccaccgtggaagatatctactgcgagggcccacccgagtacaagaagcggaagatcaacagcctgtccagcaaggacttcgactgcatcatcaccgagttcgccaagagccaggacctgccctaccagagcctgagcatcgacaccttcagctacctgaacgacgagtacgtcgtgatcgcccagccctttaccggcaagtgcatcttcctggaatgggaccacgtggaaaagaccttccggaactacgacaacatcaccggcaccagcaccgtcgtgtgcaagcccatcgtgatcgagacacagctgtacgtgatcgtggctcagctgttcggcggcagccacatctacaagcgggacagcttcgccaacaagttcatcaagatccaggacatcgagatcctgaagatccggaagcccaacgacatcgaaacctttaagattgagaacaactggtacttcgtggtggccgacagcagcaaggccggctttaccaccatctataagtggaacggcaacggcttctacagccaccagtccctgcacgcctggtacagagacaccgacgtggaatatctggaaatcgtgcggaccccccagaccctgagaacccctcacctgatcctgagcagctccagccagcggcccgtgatctaccagtggaacaaggccacccagctgtttaccaaccagaccgacatccccaacatggaagatgtgtacgccgtgaagcacttcagcgtgaagggcgacgtgtacatctgcctgacccggttcatcggcgacagcaaagtgatgaagtggggcggcagcagcttccaggatatccagcggatgcccagccggggcagcatggtgtttcagcccctgcagatcaacaactaccagtacgccatcctgggcagcgactacagcttcacccaggtgtacaattgggacgccgagaaggccaagttcgtgaagttccaggaactgaacgtgcaggcccccagatcctttacccacgtgtcaatcaacaagcggaatttcctgttcgccagctcctttaagggcaacacccagatctataagcacgtgatcgtggacctgagcgcctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2025/0144244 A1
(43) Pub. Date: May 8, 2025

(54) TREATMENT OF ACQUIRED FOCAL EPILEPSY

(71) Applicant: UCL BUSINESS LTD, London (GB)

(72) Inventors: Matthew Charles WALKER, London (GB); Gabriele LIGNANI, London (GB); Dimitri M. KULLMANN, London (GB); Eleanora LUGARÀ, London (GB); Marta PEREZ GONZALEZ, London (GB); Stephanie SCHORGE, London (GB); Albert SNOWBALL, London (GB); Jenna CARPENTER, London (GB); Elodie CHABROL-PIPARD, London (GB)

(21) Appl. No.: 18/837,652

(22) PCT Filed: Feb. 10, 2023
(86) PCT No.: PCT/EP2023/053351

§ 371 Date: Aug. 12, 2024

(30) Foreign Application Priority Data
Feb. 10, 2022 (GB) 2201744.6

(51) Int. Cl. A61K 48/00; A61K 9/00; A61K 38/17; A61P 25/08; C12N 15/86

(52) U.S. Cl. CPC A61K 48/0058; A61K 9/0085; A61K 38/1709; A61K 48/0075; A61P 25/08; C12N 15/86; C12N 2750/14143; C12N 2750/14152

(57) ABSTRACT

The invention provides methods of treatment of acquired focal epilepsy in a human subject in need of the same, the methods comprising: (i) providing an expression vector encoding a polynucleotide sequence encoding LGI1, wherein the polynucleotide sequence is operably linked to a promoter suitable to drive expression of the LGI1 in human cells, (ii) administering the expression vector to the subject. Overexpressing LGI1 has been shown to affect excitatory neurons beyond the area of expression thereby targeting a large area and affecting neurons in an advantageously uniform manner.

### CROSS-REFERENCE TO RELATED APPLICATIONS

This patent application claims the benefit of priority of GB 2201744.6 filed 10 Feb. 2022 and which is herein incorporated in its entirety.

### TECHNICAL FIELD

The present invention relates generally to methods and materials for use in the treatment of focal, acquired epilepsy.

### BACKGROUND ART

Epilepsy affects about 1% of the population. 30-40% of people with epilepsy continue to have seizures despite optimal medical treatment, and this proportion has remained unchanged over the last 30 years despite a four-fold increase in the number of medications available. For people with intractable epilepsy, surgical resection of the brain area where the seizures arise remains the best hope to achieve seizure freedom, but this procedure is only suitable for 5-10% of people with intractable epilepsy, due to difficulties identifying a discrete focus, proximity of the focus to eloquent cortex or an unacceptable impact of surgery on cognition and memory.

Gene therapy is a promising option to treat refractory focal epilepsy, but major hurdles remain.

Some previous gene therapy approaches have focused on manipulation of the potassium channel Kv1.1. For example, the gene encoding the voltage-gated potassium channel Kv1.1, KCNA1, has been codon optimized for human expression and mutated to accelerate the recovery of the channels from inactivation. For improved safety, this engineered potassium channel (EKC) gene was packaged into a nonintegrating lentiviral vector under the control of a cell type-specific CAMK2A promoter (Snowball, Albert, et al. “Epilepsy gene therapy using an engineered potassium channel.” Journal of Neuroscience 39.16 (2019): 3159-3169).

In a further example, a doxycycline-inducible CRISPRa technology has been used to increase the expression of the potassium channel gene Kcna1 (encoding Kv1.1) in mouse hippocampal excitatory neurons. CRISPRa-mediated Kv1.1 upregulation led to a substantial decrease in neuronal excitability (Colasante, Gaia, et al. “In vivo CRISPRa decreases seizures and rescues cognitive deficits in a rodent model of epilepsy.” Brain 143.3 (2020): 891-905).

It has been shown injection of shRNA-LGI1 (Leucine-rich glioma-inactivated 1) in the hippocampus increased dentate granule cell excitability and low-frequency facilitation of mossy fibers to CA3 pyramidal cell neurotransmission. Application of the Kv1 family blocker, α-dendrotoxin, occluded this effect, implicating the involvement of Kv1.1. This subacute reduction of LGI1 was also sufficient to increase neuronal network activity in neuronal primary culture (Lugarà, Eleonora, et al. “LGI1 downregulation increases neuronal circuit excitability.” Epilepsia 61.12 (2020): 2836-2846)).

However, a major limitation of Kv1.1 overexpression, as well as other gene therapy approaches, is that they only target a minority of excitatory neurons within a discrete area and are therefore not suitable for more extensive pathologies.

Furthermore, non-uniform infection of neurons by the viral vector could potentially lead to highly uneven effects on their excitability, with some neurons silenced whilst other neurons are unaffected. In addition, overexpression of Kv1.1 in principle affects areas irrespective of whether or not they are hyperexcitable.

WO2021/191474 describes expression vectors or vector systems comprising a polynucleotide sequence encoding a polypeptide, wherein the gene is operably linked to a particular neuronal activity-dependent promoter suitable to drive expression of the gene product in a subject's neural cells. The features of the expression vectors combine to advantageously improve the treatment of a neurological disorder associated with neuronal hyperexcitability in a subject.

Nevertheless it can be seen that providing novel gene therapy approaches that address one or more of the drawbacks of these previous approaches would provide a useful contribution to the art.

### DISCLOSURE OF THE INVENTION

The present inventors have shown that overexpressing a secreted protein, LGI1, can affect not only the excitatory neurons in which it is overexpressed, but also surrounding excitatory neurons. It can therefore successfully target a larger area and affect neurons in an advantageously uniform manner.

More specifically, the Examples below demonstrate that overexpressing LGI1 using an AAV vector under a CAG promoter in a rat model of mesial temporal lobe epilepsy significantly reduces seizure frequency.

It should be noted that LGI1 does not have a specific receptor and is not a channel itself. Rather it binds to other extracellular proteins and lack of it disrupts synaptic function through complex interactions. It was therefore quite unexpected that increasing its expression reduced excitability.

This therapy has at least two advantages over direct regulation of the excitability of transfected cells, for example using Kv1.1.

Firstly, LGI1 acts in a paracrine fashion to decrease the excitability of excitatory cells in a more diffuse fashion.

Secondly, the results unexpectedly suggest that LGI1 production or secretion is activity driven thereby making the therapy more effective where there is greater seizure activity, and providing the opportunity for auto regulatory gene therapy.

Based on the results described herein, the present disclosure provides novel methods and treatments for the treatment of focal acquired epilepsy.

Thus in one aspect the invention provides a method of treatment of acquired focal epilepsy in a human subject, the method comprising:
(i) providing an expression vector encoding a polynucleotide sequence encoding LGI1, wherein the polynucleotide sequence is operably linked to a promoter suitable to drive expression of the LGI1 in human cells,
(ii) administering directly or indirectly the expression vector to the subject.

In some embodiments the expression vector is provided as part of a viral vector system, for example an AAV viral vector system.

The invention further provides an expression vector or viral vector system for use in the methods of treatment of the invention.

The invention further provides use of an expression vector or viral vector system in the preparation of a medicament for use in the methods of treatment of the invention.

Some of these aspects and embodiments will now be discussed in more detail:

In focal epilepsy, seizures develop in one or more particular brain areas (or networks of brain cells). Focal seizures represent the most common seizure type and focal epilepsies the most common epilepsy type (Mula, Marco. “Pharmacological treatment of focal epilepsy in adults: an evidence based approach.” Expert Opinion on Pharmacotherapy 22.3 (2021): 317-323.)

The aetiologies of “acquired” epilepsy are diverse and result from external or environmental causes (such as traumatic brain injury or infection) as well as internal pathologic processes (such as stroke, tumour, dementia and malformations of cortical development). “Acquired epilepsy” does not exclude a genetic contribution but can be distinguished from “genetic epilepsy”, which can be considered as the direct result of a known or presumed genetic defect(s) in which seizures are the core symptom of the disorder (Berg A T, Berkovic S F, Brodie M J, Buchhalter J, Cross J H, van Emde B W, et al. Revised terminology and concepts for organization of seizures and epilepsies: report of the ILAE Commission on Classification and Terminology, 2005-2009. Epilepsia. 2010; 51:676-85).

Leucine-rich glioma inactivated protein 1 (LGI1) is a secreted brain protein and is part of the synaptic extracellular matrix (Fukata Y, Lovero K L, Iwanaga T, Watanabe A, Yokoi N, Tabuchi K, et al. Disruption of LGI1-linked synaptic complex causes abnormal synaptic transmission and epilepsy. Proc Natl Acad Sci USA. 2010; 107:3799-804).

It has previously been shown that autosomal dominant epilepsy with auditory features results from mutations in LGI1 (Seagar, Michael, et al. “LGI1 tunes intrinsic excitability by regulating the density of axonal Kv1 channels.” Proceedings of the National Academy of Sciences 114.29 (2017): 7719-7724).

As explained above it has further been shown that LGI1 downregulation increases neuronal circuit excitability, via its involvement in the trafficking and function of Kv1.1 (Lugarà, Eleonora, et al. “LGI1 downregulation increases neuronal circuit excitability.” Epilepsia 61.12 (2020): 2836-2846).

WO2021/089856 concerns the gene therapy of numerous CNS disorders including epilepsy using certain specific types of AAV vector. LGI1 is listed amongst many potential transgenes. The many epilepsy subtypes described in WO2021/089856 are genetic epilepsies, implying that the methods and materials described are intended to correct a genetic defect.

Unlike WO2021/089856, the present invention does not concern treatment of genetic epilepsy, for example that caused by mutations in LGI1 gene. The results described herein were not predicated on any genetic defect. Rather, the present invention concerns treatment of focal acquired epilepsy. WO2021/089856 does not refer at any point to the treatment of such acquired focal epilepsy.

In some embodiments, the polynucleotide sequence encoding LGI1 encodes an amino acid sequence comprising or consisting the amino acid sequence shown in SEQ ID NO: 2 or is a variant thereof.

In some embodiments, the LGI1 encodes an amino acid sequence comprising or consisting the amino acid sequence which has at least 75, 80, 85, 90, 91, 92, 93, 94, 95, 96, 97, 98, 99 or 100% sequence identity to the amino acid sequence shown in SEQ ID NO: 2.

In some embodiments, the polynucleotide sequence encoding LGI1 has a nucleotide sequence comprising, or consisting essentially of, or consisting of, the nucleotide sequence shown in SEQ ID NO: 1 or variant thereof.

For example the polynucleotide sequence encoding LGI1 may be a codon-optimised sequence such as is shown in SEQ ID NO: 3, and within the vector sequence shown in SEQ ID NO: 4

In some embodiments, the polynucleotide sequence encoding LGI1 has a nucleotide sequence comprising, or consisting essentially of, or consisting of, a nucleotide which has at least 75, 80, 85, 90, 91, 92, 93, 94, 95, 96, 97, 98, 99 or 100% sequence identity to the nucleotide sequence shown in SEQ ID NO: 1.

LGI1 variants as used in the present invention retain LGI1 activity by binding to ADAM23 and/or ADAM22, resulting in e.g. trafficking and increased synaptic expression of Kv1.1.

The treatments described herein may be used to quench or block epileptic activity. The treatments may be used to reduce the frequency of seizures. The treatments may be used to temporally reduce seizures (for example, over 2, 6, 24, 48 or 72 hours).

Alignment and calculation of percentage amino acid or nucleotide sequence identity can be achieved in various ways known to a person of skill in the art, for example, using publicly available computer software such as ClustalW 1.82, T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

The percentage identity can then be calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The term “operably linked” used herein includes the situation where a selected gene and promoter are covalently linked in such a way as to place the expression of the gene (i.e. polypeptide coding) under the influence or control of the promoter. Thus, a promoter is operably linked to a gene if the promoter is capable of effecting transcription of the gene into RNA in a cell. Where appropriate, the resulting RNA transcript may then be translated into a desired protein or polypeptide. The promoter is suitable to effect expression of the operably linked gene in a mammalian cell. Preferably, the mammalian cell is a human cell.

In one embodiment the promoter is a cell type specific promoter.

The cell type specific promoter that is used will depend on the cell type that is being targeted. For example, in the case of a treating epilepsy, it may be preferable to target neural cells, such as neurons and glial cells.

In particularly preferred examples, the cell type specific promoters is specific for neurons, in other words it drives higher levels of expression in neurons than in glial cells.

In some cases, the cell type specific promoter is specific for excitatory neurons, such as glutamatergic neurons. An example of an excitatory neuron is a pyramidal neuron. Glutamatergic neurons can be identified by detecting markers that are specific for gluatamatergic cells, such as vGlut1, vGlut2, NMDAR1, NMDAR2B, glutaminase, glutamine synthetase.

A preferred example of the neuronal cell type specific promoter is the human CAMK2A (alpha CaM kinase II gene) promoter. The CAMK2A promoter is known to bias expression to excitatory neurons and furthermore leads to very little expression in GABAergic cells (also known as interneurons) (Dittgen et al., 2004 “Lentivirus-based genetic manipulations of cortical neurons and their optical and electrophysiological monitoring in vivo.” Proc Natl Acad Sci USA 101:18206-18211; Yaguchi et al., 2013 “Characterization of the properties of seven promoters in the motor cortex of rats and monkeys after lentiviral vector-mediated gene transfer. Hum Gene Ther Methods 24:333-344”). The CAMK2A promoter is therefore an example of a cell type specific promoter that is specific for excitatory neurons.

Another promoter that is believed to be specific for neurons is the VGLUT1 promoter (Zhang et al. Brain Research 1377:1-12, 2011, herein incorporated by reference at least for the sequence of the promoters and related sequences). As described by Zhang et al., the rat VGLUT1 upstream promoter or the first intron, after fusion to a basal promoter, results in glutamatergic-specific expression. A further example of a promoter that has been shown to be specific for glutamatergic neurons in rats is the PAG promoter (Rasmussen et al. Brain Research 1144:19-32, 2007, herein incorporated by reference at least for the sequence of the promoters and related sequences).

Other neuronal cell type-specific promoters include the NSE promoter (Liu H. et al., Journal of Neuroscience. 23(18):7143-54, 2003 & Peel AL. et al., Gene Therapy. 4(1): 16-24, 1997); tyrosine hydroxylase promoter (Kessler M A. et al., Brain Research. Molecular Brain Research. 112(I-2):8-23, 2003); myelin basic protein promoter (Kessler M A. et al Biochemical & Biophysical Research Communications. 288(4):809-18, 2001); neurofilaments gene (heavy, medium, light) promoters (Yaworsky P J. et al., Journal of Biological Chemistry. 272(40):25112-20, 1997) (All of which are herein incorporated by reference at least for the sequence of the promoters and related sequences.). A further suitable promoter is the Synapsin1 promoter (see Kügler et al “Human synapsin 1 gene promoter confers highly neuron-specific long-term transgene expression from an adenoviral vector in the adult rat brain depending on the transduced area.” Gene Therapy. 10(4):337-47 2003).

In other embodiments the promoter is not a cell type specific promoter, or is not highly specific.

In one embodiment the promoter is a CAG promoter (Farokhimanesh S, Rahbarizadeh F, Rasaee M J, Kamali A and Mashkani B (2010) Hybrid promoters directed tBid gene expression to breast cancer cells by transcriptional targeting. Biotechnol Prog 26, 505-511).

CAG promoter comprises the following sequences:
(C) the cytomegalovirus (CMV) early enhancer element,
(A) the promoter, the first exon and the first intron of chicken beta-actin gene,
(G) the splice acceptor of the rabbit beta-globin gene.

The enhancer element and promoter are shown in SEQ ID NO: 4.

An expression vector as used herein is a DNA molecule used to transfer and express foreign genetic material in a cell. Such vectors include a promoter sequence operably linked to the gene encoding the protein to be expressed. “Promoter” means a minimal DNA sequence sufficient to direct transcription of a DNA sequence to which it is operably linked. “Promoter” is also meant to encompass those promoter elements sufficient for promoter-dependent gene expression controllable for cell type specific expression; such elements may be located in the 5′ or 3′ regions of the native gene. Alternatively, an expression vector may be an RNA molecule that undergoes reverse transcription to DNA as a result of the reverse transcriptase enzyme.

An expression vector may also include a termination codon and expression enhancers. Any suitable vectors, enhancers and termination codons may be used to express the gene product (LGI1) from an expression vector according to the invention. Suitable vectors include plasmids, binary vectors, phages, phagemids, viral vectors and artificial chromosomes (e.g. yeast artificial chromosomes or bacterial artificial chromosomes). As described in more detail below, preferred expression vectors include viral vectors such as AAV vectors.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. Molecular biology techniques suitable for the expression of polypeptides in cells are well known in the art. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

In one embodiment the vector is a viral vector.

A preferred expression vector for use with the present invention is a viral vector, such as a lentiviral or AAV vector. A particularly preferred expression vector is an adeno associated viral vector (AAV vector).

In some instances, the vector is a recombinant AAV vector. AAV vectors are DNA viruses of relatively small size that can integrate, in a stable and site-specific manner, into the genome of the cells that they infect. They are able to infect a wide spectrum of cells without inducing significant effects on cellular growth, morphology or differentiation. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” in Parvoviruses (J R Kerr, S F Cotmore. M E Bloom, R M Linden, C R Parrish, Eds.) p5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p15-23, Hudder Arnold, London, UK (2006), the disclosures of which are hereby incorporated by reference herein in their entireties). Methods for purifying for vectors may be found in, for example, U.S. Pat. Nos. 6,566, 118, 6,989,264, and 6,995,006 and International Patent Application Publication No.: WO/1999/011764 titled “Methods for Generating High Titer Helper-free Preparation of Recombinant AAV Vectors”, the disclosures of which are herein incorporated by reference in their entirety.

Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: WO 1/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in viva (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

Thus in one embodiment the vector is an AAV vector.

In some instances, useful AAV vectors for the expression constructs as described herein include those encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, AAV16 and AAVrh10) or pseudotypes, chimeras, and variants thereof.

In one embodiment the AAV vector is an AAV2 vector. In one embodiment the AAV vector is an AAV9 vector. In one embodiment it comprises the elements shown in the table below, optionally lacking the GFP element.

In one embodiment the vector is a vector comprising, or consisting essentially of, or consisting of, the nucleotide sequence shown in SEQ ID NO: 4 or variant thereof as described above i.e. having a nucleotide sequence comprising, or consisting essentially of, or consisting of, a nucleotide which has at least 75, 80, 85, 90, 91, 92, 93, 94, 95, 96, 97, 98, 99 or 100% sequence identity to the nucleotide sequence shown in SEQ ID NO: 4 of variant thereof.

SEQ ID NO: 4 contains the following elements:

| | Start | Finish |
|---|---|---|
| SV40 poly(A) signal | 31 | 154 |
| AAV ITR | 316 | 446 |
| ORI | 682 | 1270 |
| Ampicillin Resistance Gene | 1441 | 2301 |
| AmpR promoter | 2302 | 2406 |
| f1 ori | 2432 | 2887 |
| AAV ITR | 2958 | 3088 |
| CMV enhancer element | 3149 | 3398 |
| Chicken b actin promoter | 3399 | 3676 |
| Codon optimised LGI-1 | 4098 | 5771 |
| attB2 | 5772 | 5796 |
| IRES2 | 5792 | 6383 |
| COP-GFP | 6384 | 7050 |

One preferred variant of SEQ ID NO: 4 lacks the GFP element.

In another embodiment the promoter is a human synapsin 1 (hSyn) promoter. A plasmid containing the hSyn promoter is shown within SEQ ID NO. 5 (from base pair 163-609 inclusive).

This may for example replace the CAG promoter (CMV enhancer element/Chicken b actin promoter) in embodiments of the invention described herein e.g. SEQ ID NO: 4 optionally lacking the GFP element

In some embodiments, the viral vector additionally comprises genes encoding viral packaging and envelope proteins. As explained below, the vector (e.g. based on AAV2) may be used with a helper vector or packaged in the capsid of a different virus particle (e.g. AAV9).

In some embodiments, the viral vector is a lentiviral vector. In some embodiments, the lentiviral vector is a non-integrating lentiviral vector (NILV). Vector particles produced from these vectors do not integrate their viral genome into the genome of the cells and therefore are useful in applications where transient expression is required or for sustained episomal expression such as in quiescent cells. NILVs can be developed by mutations in the integrase enzyme or by altering the 5′ LTR and/or the 3′ LTR to prevent integrase from attaching these sequences. These modifications eliminate integrase activity without affecting reverse transcription and transport of the pre-integration complex to the nucleus. Without wishing to be bound by any particular theory, when a NILV enters a cell the lentiviral DNA is expected to remain as remains in the nucleus as an episome, leading to sustained expression in non-dividing cells (post-mitotic cells) such as neurons.

In some embodiments, the vector further comprises an AmpR gene, and/or a hGh poly(A) signal gene, and/or one or more origin of replication genes.

The term expression vector may form part of a “vector genome”, which genome may be encapsidated in a viral particle.

The invention also includes in vitro methods of making viral particles, such as lentiviral particles or adeno-associated viral particles. In one embodiment, this method involves transducing mammalian cells with a viral vector as described herein and expressing viral packaging and envelope proteins necessary for particle formation in the cells and culturing the transduced cells in a culture medium, such that the cells produce viral particles that are released into the medium. An example of a suitable mammalian cell is a human embryonic kidney (HEK) 293 cell.

It is possible to use a single expression vector that encodes all the viral components required for viral particle formation and function. Most often, however, multiple plasmid expression vectors or individual expression cassettes integrated stably into a host cell are utilised to separate the various genetic components that generate the viral vector particles.

In some embodiments, expression cassettes encoding the one or more viral packaging and envelope proteins have been integrated stably into a mammalian cell. In these embodiments, transducing these cells with a viral vector described herein is sufficient to result in the production of viral particles without the addition of further expression vectors.

In other embodiments, the in vitro methods involve using multiple expression vectors. In some embodiments, the method comprises transducing the mammalian cells with one or more expression vectors encoding the viral packaging and envelope proteins that encode the viral packaging and envelope proteins necessary for particle formation.

Examples of suitable viral packaging and envelope proteins and expression vectors encoding these proteins are commercially available and well known in the art. In general, the viral packaging expression vector or expression cassette expresses the gag, pol, rev, and tat gene regions of HIV-1 which encode proteins required for vector particle formation and vector processing. In general, the viral envelope expression vector or expression cassette expresses an envelope protein such as VSV-G. In some cases, the packaging proteins are provided on two separate vectors—one encoding Rev and one encoding Gag and Pol. Examples of lentiviral vectors along with their associated packaging and envelope vectors include those of Dull, T. et al., “A Third-generation lentivirus vector with a conditional packaging system” J. Virol 72(11):8463-71 (1998), which is herein incorporated by reference.

The ssDNA AAV genome contains two open reading frames, Rep and Cap, flanked by two 145 base inverted terminal repeats (ITRs) fundamental for the synthesis of the complementary DNA strand. Rep and Cap produce multiple proteins (Rep78, Rep68, Rep52, Rep40, which are required for the AAV life cycle; and VP1, VP2, VP3, which are capsid proteins). The transgene will be inserted between the ITRs and Rep and Cap in trans. An AAV2 backbone is commonly used and is described in Srivastava et al., J. Virol., 45:555-564 (1983). Cis-acting sequences directing viral DNA replication (ori), packaging (pkg) and host cell chromosome integration (int) are contained within the ITRs. AAVs also require a helper plasmid containing genes from adenovirus. These genes (E4, E2a and VA) mediate AAV replication. An example of a pAAV plasmid is available from Addgene (Cambridge, MA, USA) as plasmid number 112865 or 60958.

Following release of viral particles, the culture medium comprising the viral particles may be collected and, optionally the viral particles may be separated from the culture medium. Optionally, the viral particles may be concentrated.

Following production and optional concentration, the viral particles may be stored, for example by freezing at −80° C. ready for use by administering to a cell and/or use in therapy.

Accordingly, the instant disclosure includes a recombinant virus particle (recombinant because it contains a recombinant polynucleotide) comprising any of the LGI1 expression vectors described herein.

In one embodiment the method comprises the steps of:
(i) assembling viral particles in vitro by transducing mammalian cells with the expression vector and expressing viral packaging and envelope proteins necessary for particle formation in the cells and culturing the transduced cells in a culture medium, such that the cells produce viral particles comprising the expression vector that are released into the medium;
(ii) administering the viral particles to the subject.

The method may further comprise transducing the mammalian cells with one or more viral packaging and envelope expression vectors that encode the viral packaging and envelope proteins necessary for particle formation.

In one embodiment the vector or viral particle does not comprise surface-bound saccharides e.g. of the type described in WO2021/089856. Those saccharides may be selected from the group comprising monosaccharides, oligosaccharides and polysaccharides e.g. may be a hexose, preferably a mannose, galactose or N-acetylglucosamine.

The viral particles and expression vectors described herein can be delivered to the subject in a variety of ways, such as direct injection of the viral particles into the brain. For example, the treatment may involve direct injection of the viral particles into the cerebral cortex, in particular the neocortex or hippocampal formation. Another site of injection is an area of cortical malformation or hamartoma suspected of generating seizures, as occurs in focal cortical dysplasia or tuberous sclerosis. The treatment may involve direct injection of the viral particles into the location in the brain where it is believed to be functionally associated with the disorder. For example, where the treatment is for myoclonic epilepsy this may involve direct injection of the viral particles into the motor cortex; where the treatment is for chronic or episodic pain, this may involve direct injection of the viral particles into the dorsal root ganglia, trigeminal ganglia or sphenopalatine ganglia; and where the treatment is for Parkinson's disease, this may involve direct injection of the viral particles into the substantia nigra, subthalamic nucleus, globus pallidus or putamen. The particular method and site of administration would be at the discretion of the physician who would also select administration techniques using his/her common general knowledge and those techniques known to a skilled practitioner.

The invention may also be used to treat multiple epileptic foci simultaneously by injection directly into the multiple identified loci.

In one embodiment, the expression vector or viral particles comprising the expression vector are administered directly to the hippocampus in the subject.

In one embodiment, the expression vector or viral particles comprising the expression vector are administered through injection into brain parenchyma, for example either via burr holes or a craniotomy.

In one embodiment, the expression vector or viral particles comprising the expression vector are administered directly to an area of neocortex in the subject.

In one embodiment, the expression vector or viral particles comprising the expression vector are administered directly to multiple cortical areas in the subject.

In one embodiment, the expression vector or viral particles comprising the expression vector are administered directly to subcortical areas in the subject.

In some embodiments the LGI1 expressed by the vector affects not only the excitatory neurons in which it is overexpressed, but also surrounding excitatory neurons. It can therefore successfully target a larger area and affect neurons more uniformly. In some embodiments the level of expression of the LGI1 by the vector increases when the excitatory neuron becomes more excited and decreases when the neuron becomes less excited.

As explained above, the present invention relates to types of focal acquired epilepsy, and does not concern generalized epilepsy or genetic epilepsy, for example that are caused specifically by mutations in LGI1 gene.

As explained above, focal epilepsies include those that result from external or environmental causes (such as traumatic brain injury or infection) as well as internal pathologic processes (such as stroke, tumour, dementia and malformations of cortical development).

In one embodiment the invention concerns the treatment of well defined focal epilepsy affecting a single area of the brain.

However, the gene therapy treatment is also suitable patients with seizures arising from several parts of the brain. The invention may be used with seeking to cease taking anti-epileptic drugs.

In one embodiment the invention concerns the treatment of diffuse pathologies in which a larger area and number of neurons need to be targeted, which may be achieved through a plurality (e.g. 2, 3 or 4) cortical injections.

In one embodiment the invention concerns the treatment of temporal lobe epilepsy with hippocampal sclerosis. Hippocampal sclerosis is the commonest cause of drug-resistant epilepsy). The patient may be one who has been diagnosed as having drug-resistant or medically-refractory epilepsy, by which is meant that epileptic seizures continue despite adequate administration of antiepileptic drugs.

Following administration of the viral particles, it is intended that the recipient individual exhibits a reduction in symptoms. In the present context, the recipient individual may exhibit a reduction in the frequency or severity of seizures.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The viral particle can be delivered in a therapeutically-effective amount.

The term “therapeutically-effective amount” as used herein, pertains to that amount of the viral particle which is effective for producing some desired therapeutic effect, commensurate with a reasonable benefit/risk ratio, when administered in accordance with a desired treatment regimen.

Similarly, the term “prophylactically effective amount,” as used herein pertains to that amount of the viral particle which is effective for producing some desired prophylactic effect, commensurate with a reasonable benefit/risk ratio, when administered in accordance with a desired treatment regimen.

“Prophylaxis” in the context of the present specification should not be understood to describe complete success i.e. complete protection or complete prevention. Rather prophylaxis in the present context refers to a measure which is administered in advance of detection of a symptomatic condition with the aim of preserving health by helping to delay, mitigate or avoid that particular condition.

While it is possible for the viral particle to be used (e.g., administered) alone, it is often preferable to present it as a composition or formulation e.g. with a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition (e.g., formulation, preparation, medicament) comprising, or consisting essentially of, or consisting of as a sole active ingredient, viral particle as described herein, and a pharmaceutically acceptable carrier, diluent, or excipient.

As described in WO2008096268, in gene therapy embodiments employing delivery of the viral particle, the unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100 fold) due to the presence of infection-defective particles.

In some embodiments the methods or treatments of the present invention may be combined with other therapies, whether symptomatic or disease modifying.

The term “treatment” includes combination treatments and therapies, in which two or more treatments or therapies are combined, for example, sequentially or simultaneously.

For example it may be beneficial to combine treatment with a compound as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the diseases described herein, subject to the diagnosis of the individual being treated. For example epilepsy can sometimes be ameliorated by directly treating the underlying etiology, but anticonvulsant drugs, such as phenytoin, gabapentin, lamotrigine, levetiracetam, carbamazepine, clobazam, topiramate, and others, which suppress the abnormal electrical discharges and seizures, are the mainstay of conventional treatment (Rho & Sankar, 1999, Epilepsia 40: 1471-1483).

The particular combination would be at the discretion of the physician who would also select dosages using his/her common general knowledge and dosing regimens known to a skilled practitioner.

The agents (i.e. viral particle, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The expression vectors or viral vectors for use described herein may be provided as a kit or kit-of-parts suitable for transducing a cell in the central nervous system (CNS) of a subject; and/or delivering the LGI1 transgene to the central nervous system (CNS) of a subject, in each case for treating focal acquired epilepsy in a subject, said kit comprising:
(a) the expression vector or viral particle;
(b) a device for CNS delivery of the the expression vector or viral particle;
(c) optionally, instructions for CNS delivery of the the expression vector or viral particle, for use in treating focal acquired epilepsy.

Such kits may comprise, in addition to an expression vector or particle of the invention, one or more viral packaging and envelope expression vectors that encode viral packaging and envelope proteins necessary for particle formation when expressed in a cell. Optionally, the viral packaging expression vector is an integrase-deficient viral packaging expression vector.

In other aspects the invention provides:

An expression vector or viral particle comprising an expression vector for use in a method of treatment of acquired focal epilepsy in a human subject as described herein.

A method of treatment of acquired focal epilepsy in a human subject comprising use of an expression vector or viral particle comprising an expression vector as defined herein.

Use of an expression vector or use of a viral particle comprising an expression vector as defined herein in the manufacture of a medicament for the treatment of acquired focal epilepsy in a human subject.

Use of an expression vector or use of a viral particle comprising an expression vector in the manufacture of a medicament for the treatment of acquired focal epilepsy in a human subject, wherein the treatment is as described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### SEQUENCES

SEQ ID Nos 1-4 are described in the Annex hereinafter.

### FIGURES

FIG. 1: Schematic illustration of LGI1 activity in pre-and post-synaptic terminals.

FIG. 2: Over-expression of LGI1. We used two batches of rats. Each rat had continuous wireless EEG. Epilepsy was induced following repeated injections with kainic acid. After 3 weeks: the rats were randomized to injection with either an AAV vector solely expressing Green Fluorescent Protein or AAV expressing LGI1 under a CAG promoter into the dorsal hippocampus. After two weeks during which the protein is expressed, seizures were then monitored for a further 5 weeks. The animals were then killed at the end of the experiment and Western blots were used to assess the increase in LGI1 expression compared to controls. There was a significant increase in LGI1 in the treated animals.

FIG. 3: LGI1 over-expression reduces seizure frequency. We analysed seizure frequency in control animals and in all treated animals in which there was a >3 fold increase in LGI1 expression over 5 weeks. Box and whisker plot of normalized seizure frequencies to baseline. There was a significant (P<0.05) reduction in seizure frequency in the LGI1 treated animals compared to control.

FIG. 4: LGI1 over-expression reduces seizure duration. Two mice treated with a control virus expressing green fluorescent protein (AAV9-hSyn-eGFP) exhibited an increase in mean seizure duration (‘Control’), while all 5 mice treated with a virus overexpressing LGI1 (AAV9-hSyn-{codon optimised}hLGI1-IRES2-eGFP) [see SEQ ID NO: 5] exhibited a decrease in seizure duration (‘LGI1’). The difference in seizure duration was significant at p=0.007 (Student's t test).

FIG. 5: High network activity increases LGI1 expression. We transfected cultured neurons with AAV-LGI1 and then using Western Blot quantified the LGI1 present in the cytoskeleton in artificial cerebrospinal fluid (aCSF) or following application of high potassium to increase network activity. There was a significant (P<0.05) increase in LGI1 under conditions of increased network activity.

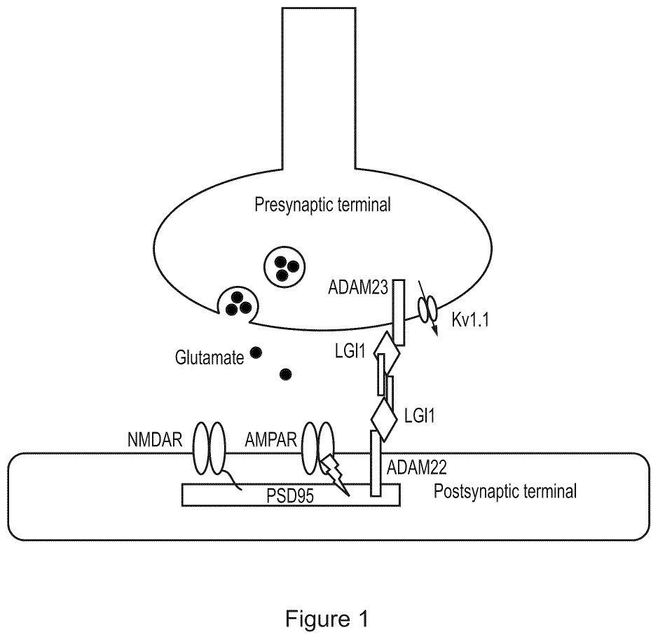

### EXAMPLE 1

To date the academic team have generated a construct comprising an AAV vector (AAV9 vector) to deliver the LGI1 gene under a CAG promoter. In a rat model of mesial temporal lobe epilepsy, the construct has been shown to significantly increase levels of LGI1 and significantly reduce seizure frequency. It is believed that overexpressing LGI1 affects not only the excitatory neurons in which it is overexpressed, but also surrounding excitatory neurons. It can therefore successfully target a larger area and affect neurons more uniformly, potentially overcoming some of the limitations associated with Kv1.1 overexpression.

### Material and Methods

### Plasmid Generation

AAV plasmids were created using standard subcloning techniques. LGI1 was codon optimized for human expression using GeneOptimizer software and was synthesized using GeneArt (Thermo Fisher Scientific). All plasmids were fully sequenced before use.

### Plasmid on HEK Cells

The recombinant AAV9 (rAAV9) LGI1 and GFP plasmids were tested on HET293T cell lines for expression of GFP fluorescent protein. HEK 293 cells (˜70% confluence) were transfected o/n with a mixture of 2.5 μg of AAV-CAG1-hLGI1-ires-dscGFP DNA and 5 μl lipofectamin2000@ into 1000 μl of new media in each of the 35 mm well. The next morning the media was replaced with fresh Optimem (Thermo Fisher) and 24 hr afterwards the media was collected, spin down to remove cellular debris (1000 rpm, 2 minutes), filtered by centrifugation with Amicon Ultra-15 Centrifugal Filter Unit (Merck, >30 KDa) at 4000 rpm for 15 minutes (4° C.) and stored in −80° C. until used. The cells were then used for in vitro fixation, cells were quickly washed with sterile PBS and then fixed in 4% PFA solution for 20 minutes at RT. Coverslips were washed 3 times with PBS solution, permeabilised with 0.1% Triton x solution for 10 minutes, then washed 3×5 minutes with PBS and incubated for 30 minutes with blocking solution and a nuclear stain (Hoechst 33342) was applied. A last 3×5 minutes wash was done before the coverslips were mounted upside-down on a droplet of mounting media (Sigma Aldrich) on a glass microslide (VWR). GFP fluorescence was visualized with a Zeiss microscope.

### Virus Production and In Vivo Testing

AAV9-CAG-LGI1-IRES-dscGFP (>10¹³ GC/ml, GC=genome copies) and control vector (AAV9-CAG-dscGFP, 1.83¹² GC/ml) were commercially produced by VectorBuilder in an AAV2 backbone constructed in house.

The viruses were initially tested in mice pups (post-natal day 1) and adult rats. The pups were put asleep with cryoanaesthesia and injecting 2 μl of virus at maximum concentration in each ventricle with a Hamilton syringe. The pups were then left to recover and sacrificed 3 weeks later with terminal anesthesia. Male Sprague-Dawley rats (300 g) were anaesthetised with isoflurane (5% in 2 L/min O2), the animal's head was shaved and then placed in a stereotaxic frame (Kopf Instruments, USA). Eyegel was applied on the animal's eye, and the animal was injected with Metacam (1.3 mg/kg) and Buprenorphine (0.2 mg/kg) subcutaneously. The head's skin was cleaned with iodine and the skull exposed. Isoflurane was then decreased to 2-3% in 2 L/min O2 and the animal's breath was monitored through all the procedure to avoid gasping. One burr hole was unilaterally drilled according to the coordinates from bregma suture: dorsal hippocampus (Medio-Lateral [ML]—2.5; Antero Posterior [AP]—3.6; Dorso-Ventral [DV]—3.00) and ventral hippocampus (ML—5.00; AP—5.30; DV1—7.10; DV2—4.40). 2×2 μl of each virus was injected bilaterally using a Hamilton syringe at the speed of 100 nL/min and waited 5-10 min before slowly withdrawing the micropipette to avoid backflow of the virus to the surface. The skin was then sewed, saline solution (2.5 ml) was administered, and the animal was monitored until awake. Three weeks after injection the Cortex (pups) and hippocampus (rats) were extracted snap frozen and prepared for western blot.

A further round of experiments tested the anti-seizure effect in mice. Status epilepticus was induced in mice by injecting kainic acid (KA) into the right amygdala as previously described (Colasante, et al 2020, supra). A wireless electrocorticogram transmitter (Open Source Instruments) was implanted two weeks later, with a recording electrode over the right somatosensory cortex and a contralateral reference electrode. A baseline was recorded for 2 weeks to assess spontaneous seizures. Animals that exhibited seizures were then randomised for treatment with AAV9-hSyn--{codon optimised}hLGI1-IRES2-eGFP or AAV9-hSyn-eGFP, delivered at three coordinates in the right hippocampus (200 nl at 10¹² vgml). After waiting three weeks for viral expression, the electrocorticogram was recorded again for up to two weeks. Seizures were detected with a classifier based on a supervised learning algorithm, and the start and end of each seizure was annotated manually, whilst blind to the treatment group.

### Generation of Kainate Rats and EEG Implantation and Recordings

Kainic acid 50 mg/ml stock powder (KA, Tocris Bioscience) was dissolved in 0.9% sterile saline solution for a single dose of 5 mg/kg for each injection. Rats were injected intraperitoneally once every 30 minutes until they reached stage 5 of a modified Racine scale or reached a maximum dose of 45 mg/kg. Once that the rats reached stage 5 for a consistent amount of time (90-120 minutes) Diazepam (10 mg/kg) was injected SC. Rats were then left single caged for 10-12 weeks, at which point cortical EEG transmitters (OSI system) were stereotactically implanted together with bilateral guide cannulas on top of the two hippocampi. After 3 weeks baseline recordings, the rats were randomised to LGI1 therapy or control virus. Viruses were injected as described above and the EEG was continuously recorded for 7 weeks. Traces were acquired using a A3028E telemetry transmitter (0.3-160 Hz, 256 samples/s) and video recordings. Data were analysed using a bespoke software, where number of motor seizures (Racine stages 3 to 5) were counted for the data analysis. At the end of the recording, the hippocampi were extracted snap frozen and prepared for western blot.

### Western Blot

The samples were lysed in RIPA buffer (Radioimmunoprecipitation assay buffer) (150 mM sodium chloride, 1.0% Triton X-100, 0.5% sodium deoxycholate, 0.1% sodium dodecyl sulfate, 50 mM Tris, pH 8.0. Sigma Aldrich) and protease inhibitor was added to the lysis mixture following the recommended concentration (Thermo Fisher). Tissue/cells were initially mechanically disrupted by manual up and down with a pipette and then processed by a mechanical rotor type homogenizer (FastPrep-24, MP Biomedicals LLC) with the use of homogenizer beads (SLS Scientific Laboratory Supplies). The samples were then centrifuged, and the pellet discarded. Samples were run for 3 hr at 70V and then transferred to a nitrocellulose membrane by semi-dry method and blocked with 5% BSA and then 3% milk in PBS. Membranes were incubated with 1:250 anti-LGI1 in PBS (Santa Cruz c-19), 1:2000 anti β-actin in PBS (Sigma) o/n. The secondary HRP antibody used were donkey anti-goat (1:2500, Santa Cruz) and donkey anti-mouse (1:4000, 2B scientific) in PBS and were incubated for 1.30 h at RT shaking. Thermo Scientific PierceM ECL substrate (#32106) was used for the detection. Pictures were taken by a ChemiDoc™ Imaging System (BioRad) and analysed with Image Lab™ software (BioRad) for quantification of volume intensity of the revealed bands. LGI1 was normalised over beta-actin of the same sample lane.

### Statistics

The Western blots were normalized by a control protein (β-actin) and the quantification for LGI1-treated animals were normalised by controls. Average seizure frequency from week 3-7 following viral vector injection were normalized to the baseline seizure frequency (week 1-2 were not included to permit vector expression). The results from treated animals were compared to those of controls using unpaired Student's t tests or Mann-Whitney U, as appropriate, with P<0.05 considered significant. Seizure frequency was only analysed in animals in which there was 3 fold or greater increase in LGi1 expression.

### Results

A codon-optimised human LGI1 with an AAV2 backbone and GFP reporter under a CAG promoter were packaged in a AAV9 capsid. A control vector was also produced which expressed GFP under the same promoter but lacked LGI1. Vector expression was confirmed in three mice pups through intraventricular injection and two rats through intrahippocampal injection and Western Blot analysis confirmed increased expression of LGI1 in animal injected with the vector carrying LGI1.

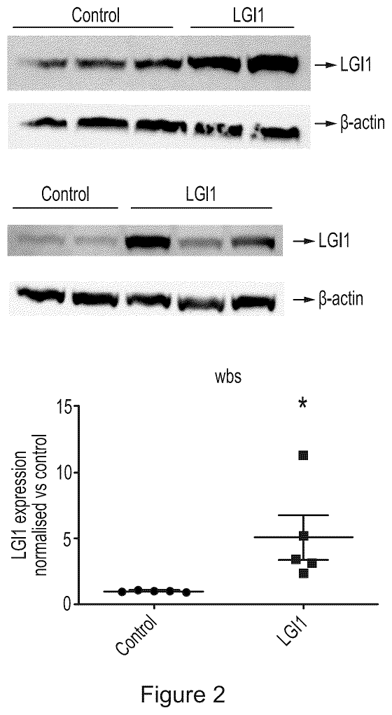

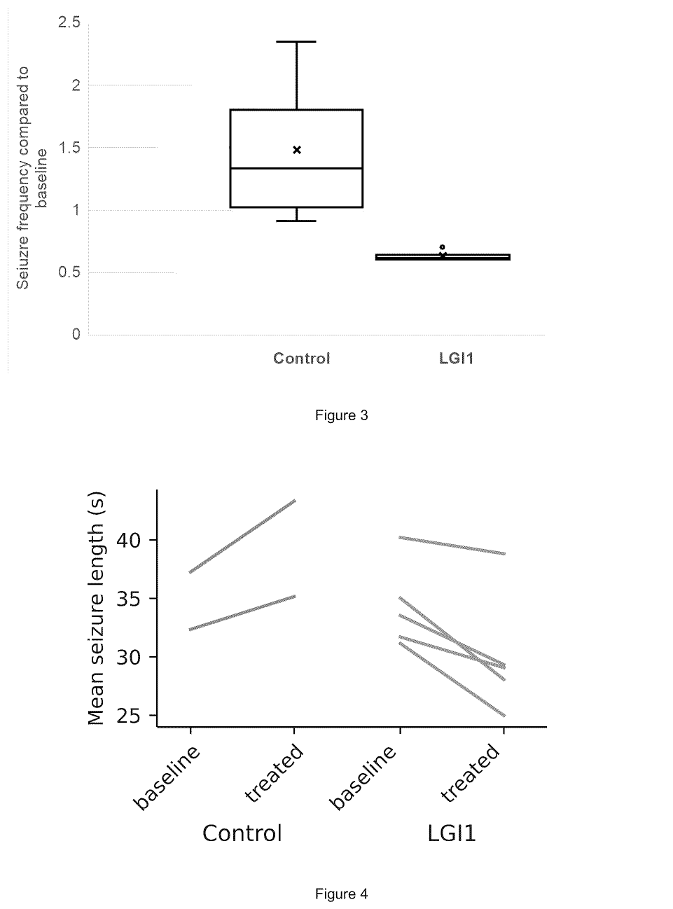

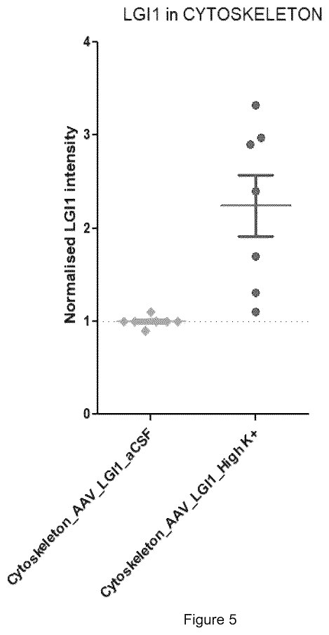

We next asked if overexpression of LGI1 could reduce seizure frequency in an animal model of hippocampal sclerosis. We used repeated systemic injections of kainic acid to induce status epilepticus, which was then terminated with diazepam; these animals develop chronic temporal lobe epilepsy. At 10-12 weeks, the animals were monitored with continuous video-EEG for three weeks at which point they were randomized to receive control vector or LGI-vector bilaterally into the hippocampus and were monitored continuously with video-EEG telemetry. Seven weeks after vector injection, the animals were killed and using Western blot analysis there was on average a greater than five-fold increase in hippocampal LGI1 expression compared to controls (FIG. 2, n=5 in each group, P<0.05, Mann-Whitney U test). All but one of the treated animals had a greater than 3-fold increase in LGI1 expression. We then compared the seizures in the rats with a >3 fold expression of LGI1 to control from 3-7 weeks after vector injection to allow a two week period for vector expression. The control animals (n=5) demonstrated a 1.5±0.3 fold increase in seizure frequency whilst the LGI1 treated animals (n=4) demonstrated a 0.6±0.2 fold decrease in seizure frequency (FIG. 3; P<0.05 for difference). There was an overall reduction in the LGI1 animals in seizure frequency compared to baseline (P<0.01). Thus, increasing the expression of LGI1 in the hippocampi of animals with temporal lobe epilepsy reduces seizure frequency.

LGI1 overexpression was also tested in a mouse model of temporal lobe epilepsy induced by intra-amygdala kainate injection. The duration of each seizure was determined whilst blind to the treatment group. LGI1 treatment significantly decreased average seizure duration in comparison with treatment with the control virus expressing GFP alone (p=0.007, Student's t test).

### EXAMPLE 2

Summary: data generated in which neurons were transfected in culture with LGI1 using AAV demonstrated that LGI1 produced is activity dependent. This increase of LGI1 in an activity-dependent manner suggests an auto regulatory gene therapy. This was an unexpected effect.

### Material and Methods

### Primary Neuronal Cultures and MEA Preparation

Pregnant Sprague Dawley rats were ordered from Charles River UK Ltd one week prior to dissection. Dissection occurred between embryonic day 17 (E17) and E19. The pregnant rat was culled in a CO₂ chamber. To confirm death, the spinal cord was severed mechanically. The rat was placed in a sterile dissection room and its abdomen was sterilized with 70% ethanol. Sterile dissection tools were used to cut open the skin and abdominal lining. Embryos were removed and placed in Falcons of Hibernate-E (Gibco) (on ice). Extracted embryos were transferred to a sterile petri dish containing cold Hank's Balanced Salt Solution (HBSS 1×, Modified, Sigma). Sterile dissection tools were used to remove the embryos from their amniotic sacs. The heads were removed and placed in a second sterile petri dish with cold HBSS. The brains were gently removed from the skull and placed in a third sterile petri dish with cold HBSS. Each hemisphere was cut from the brain. For each hemisphere, the meninges were removed. The cortices were dissected out and washed several times with cold HBSS and once with DMEM-FBS (DMEM 1×, Gibco+10% FBS, Gibco) before insertion into a new 1 mL of HBSS for resuspension. All cells were resuspended by pipetting 7-8 times using pipette tip rinsed with DMEM-FBS. Samples were placed on ice for 5 minutes. The supernatant was transferred to a new sterile Falcon. Cells were centrifuged at RT for 3 minutes at 1500 rpm. The pellet was resuspended in 1mL DMEM-FBS. Cell counting was performed using a haemocytometer (Bright Line, 0.1 mm deep). Cells were plated at the desired concentrations onto plates or coverslips that had been treated with Poly-L-Lysine (PLL) at 30,000-70,000 MW or 70,000-150,000 MW (Poly-L-lysine hydrobromide, Sigma).

### MEAs Microelectrode Arrays (MEAs)

60-6 well MEA200/30iR-Ti arrays (Multichannel Systems) were used with 21 DIV neurons for recording. MEAs were FBS treated before PLL coating. Plated samples (60 k cells/well) were placed in the 37° C. incubator for 3 hours. Media was changed to warmed Neurobasal Complete [NB++] (Neurobasal Medium 1× from Gibco+1% Gibco Penstrep+1% Gibco Glutamax+2% Gibco B27 supplement) and samples were returned to the incubator. During recording, the MEAs were maintained at 37° C. using a temperature controller (Multichannel Systems, TC01 1-channel temperature controller). Baseline recordings were taken for 5 minutes in aCSF before changing the media to High K+ buffer for half of the wells. The other half were left in aCSF. MEAs were recorded for 30 minutes (5-minute intervals) following media changes. Data was analyzed using MATLAB (MATLAB R2016b, The MathWorks), SpyCode (SpyCode v3.8, NBT-NeuroTech Group@IIT, for MATLAB), and GraphPad (GraphPad Prism 6, Version 6.01) softwares.

### 7 Propidium Iodide Mortality Experiments

21 DIV cortical neurons onto 25 mm diameter glass coverslips were incubated for varying times in High K+ buffer. Samples were concurrently incubated in propidium iodide dye (1:1000) (Sigma-Aldrich) and Hoechst dye (1:1333) (Thermo Scientific, Hoechst 33342) for 20 minutes. Baseline samples were incubated in aCSF. Samples were subsequently placed in a coverslip holder and aCSF was pipetted onto each coverslip. They were imaged using a Zeiss confocal microscope (Zeiss LSM 710) at 20× magnification. Counting and analysis was performed using Fiji ImageJ software (ImageJ, open source software) and GraphPad (GraphPad Prism 6, Version 6.01).

### Western Blot

Neuronal cells were used at ˜21 days in vitro (DIV) at 2,500,000 cells/mL. The media was aspirated and replaced with aCSF or High K+ solution, [aCSF (in mM) =CaCl₂ (2), MgCl₂ (1), HEPES (10), D-Glucose (10), NaCl (140), KCl (4)] [High K+ (in mM) =CaCl₂ (2), MgCl₂ (0 or 1), HEPES (10), Glucose (10), NaCl (140), KCl (50, 15, or 10)] and incubated. This media was removed and kept in a Falcon on ice. Media was centrifuged (4° C., 2000 rpm, 2-3 minutes) and supernatant was collected on ice. The cells were collected using RIPA buffer with a protease inhibitor cocktail (Complete Mini Protease Inhibitor

Cocktail Tablets, Roche Diagnostics) and were placed on ice. Media supernatant was placed in Centrifuge Filter Spin Tubes (Millipore, 30,000 MW). Media samples were spun down 4° C. at 5000 rpm until the volume remaining was less than 200 uL. This final volume was tested for concentration using Bradford Assays. The cell samples were centrifuged was RT at 14680 rpm for 10 minutes. The supernatants were transferred into new eppendorfs and used for Bradford Assays. Later experiments used a protein extraction kit (Compartmental Protein Extraction Kit, Millipore) to extract neuronal samples as fractions of the intracellular and extracellular space. All samples were processed by Bradford Assay once collected.

### Results

The first step was to implement and test a model of seizure-like activity in a network using in vitro cultures. For this reason, cortical cultures were plated on a MEA system (multielectrode arrays) which allows growing the cortical cultures while still retaining the ability to record the electrical activity. 21 DIV after the plating, the cells were recorded for 5 minutes for baseline activity and the for 30 minutes following media change with either aCSF or the high potassium one as described in the methods (15 mM K+ and no Magnesium). Neurons treated with the high potassium medium were bursting more than those treated with normal aCSF. Second, we checked the viability of the neuronal cells treated with high potassium using the priopidium iodide staining by live imaging. Neuronal survival was not affected for the 30 minutes of the experiment.

We next asked if this increase in activity results in increased LGI1 in cultures transfected with our AAV-LGI gene therapy. In 15 mM High K+ (no Mg++) samples transduced with AAV-LGI1 there was a >2 fold increase in the cytoskeletal fraction, compared to that in control aCSF (P<0.05). Smaller amounts of LGI1 were also visible in the membrane fraction of high potassium samples. No LGI1 was detected in AAV-GFP samples.

Sequence annex

SEQ ID NO: 1-LGI1 nucleotide sequence
ATGGAATCAG AAAGAAGCAA AAGGATGGGA AATGCCTGCA TTCCCCTGAA AAGAATTGCT
TATTTCCTAT GTCTCTTATC TGCGCTTTTG CTGACTGAGG GGAAGAAACC AGCGAAGCCA
AAATGCCCTG CCGTGTGTAC TTGTACCAAA GATAATGCTT TATGTGAGAA TGCCAGATCC
ATTCCACGCA CCGTTCCTCC TGATGTTATC TCATTATCCT TTGTGAGATC TGGTTTTACT
GAAATCTCAG AAGGGAGTTT TTTATTCACG CCATCGCTGC AGCTCTTGTT ATTCACATCG
AACTCCTTTG ATGTGATCAG TGATGATGCT TTTATTGGTC TTCCACATCT AGAGTATTTA
TTCATAGAAA ACAACAACAT CAAGTCAATT TCAAGACATA CTTTCCGGGG ACTAAAGTCA
TTAATTCACT TGAGCCTTGC AAACAACAAT CTCCAGACAC TCCCAAAAGA TATTTTCAAA
GGCCTGGATT CTTTAACAAA TGTGGACCTG AGGGGTAATT CATTTAATTG TGACTGTAAA
CTGAAATGGC TAGTGGAATG GCTTGGCCAC ACCAATGCAA CTGTTGAAGA CATCTACTGC
GAAGGCCCCC CAGAATACAA GAAGCGCAAA ATCAATAGTC TCTCCTCGAA GGATTTCGAT
TGCATCATTA CAGAATTTGC AAAGTCTCAA GACCTGCCTT ATCAATCATT GTCCATAGAC
ACTTTTTCTT ATTTGAATGA TGAGTATGTA GTCATCGCTC AGCCTTTTAC TGGAAAATGC
ATTTTCCTTG AATGGGACCA TGTGGAAAAG ACCTTCCGGA ATTATGACAA CATTACAGGC
ACATCCACTG TAGTATGCAA GCCTATAGTC ATTGAAACTC AGCTCTATGT TATTGTGGCC
CAGCTGTTTG GTGGCTCTCA CATCTATAAG CGAGACAGTT TTGCAAATAA ATTCATAAAA
ATCCAGGATA TTGAAATTCT CAAAATCCGA AAACCCAATG ACATTGAAAC ATTCAAGATT
GAAAACAACT GGTACTTTGT TGTTGCTGAC AGTTCAAAAG CTGGTTTTAC TACCATTTAC
AAATGGAACG GAAACGGATT CTACTCCCAT CAATCCTTAC ACGCGTGGTA CAGGGACACT
GATGTGGAAT ATCTAGAAAT AGTCAGAACA CCTCAGACAC TCAGAACGCC TCATTTAATT
CTGTCTAGTA GTTCCCAGCG TCCTGTAATT TATCAGTGGA ACAAAGCAAC ACAATTATTC
ACTAACCAAA CTGACATTCC TAACATGGAG GATGTGTACG CAGTGAAGCA CTTCTCAGTG
AAAGGGGACG TGTACATTTG CTTGACAAGA TTCATTGGTG ATTCCAAAGT CATGAAATGG
GGAGGCTCCT CGTTCCAGGA TATTCAGAGG ATGCCATCGC GAGGATCCAT GGTGTTCCAG
CCTCTTCAAA TAAATAATTA CCAATATGCA ATTCTTGGAA GTGATTACTC CTTTACTCAA
GTGTATAACT GGGATGCAGA GAAAGCCAAA TTTGTGAAAT TTCAGGAATT AAATGTTCAG
GCACCAAGAT CATTCACACA TGTGTCCATT AATAAGCGTA ATTTTCTTTT TGCTTCCAGT
TTTAAGGGAA ATACACAGAT TTACAAACAT GTCATAGTTG ACTTAAGCGC ATGA*
SEQ ID NO: 2-LGI1 amino acid sequence
MESERSKRMG NACIPLKRIA YFLCLLSALL LTEGKKPAKP KCPAVCTCTK
DNALCENARS IPRTVPPDVI SLSFVRSGFT EISEGSFLFT PSLQLLLFTS
NSFDVISDDA FIGLPHLEYL FIENNNIKSI SRHTFRGLKS LIHLSLANNN
LQTLPKDIFK GLDSLINVDL RGNSFNCDCK LKWLVEWLGH TNATVEDIYC
EGPPEYKKRK INSLSSKDFD CIITEFAKSQ DLPYQSLSID TFSYLNDEYV
VIAQPFTGKC IFLEWDHVEK TFRNYDNITG TSTVVCKPIV IETQLYVIVA
QLFGGSHIYK RDSFANKFIK IQDIEILKIR KPNDIETFKI ENNWYFVVAD
SSKAGFTTIY KWNGNGFYSH QSLHAWYRDT DVEYLEIVRT PQTLRTPHLI
LSSSSQRPVI YQWNKATQLF TNQTDIPNME DVYAVKHFSV KGDVYICLTR
FIGDSKVMKW GGSSFQDIQR MPSRGSMVFQ PLQINNYQYA ILGSDYSFTQ
VYNWDAEKAK FVKFQELNVQ APRSFTHVSI NKRNFLFASS FKGNTQIYKH
VIVDLSA*
SEQ ID NO: 3-codon optimised LGI1 nucleotide sequence
ATGGAAAGCGAGCGGAGCAAGCGGATGGGCAACGCCTGCATCCCCCTGAAGCGGATCGCCTACTTC
CTGTGCCTGCTGAGCGCCCTGCTGCTGACCGAGGGAAAGAAGCCCGCCAAGCCTAAGTGCCCTGCC
GTGTGCACCTGTACCAAGGACAACGCCCTGTGCGAGAACGCCCGGTCCATCCCTAGAACCGTGCCC
CCCGATGTGATCAGCCTGAGCTTTGTGCGGAGCGGCTTCACCGAGATCAGCGAGGGCAGCTTCCTG
TTCACCCCCAGCCTGCAGCTGCTGCTGTTCACCAGCAACAGCTTCGACGTGATCAGCGACGACGCC
TTCATCGGCCTGCCCCACCTGGAATACCTGTTCATCGAGAACAACAACATCAAGAGCATCAGCCGG
CACACCTTCCGGGGCCTGAAGTCCCTGATCCACCTGAGCCTGGCCAACAACAACCTGCAGACCCTG
CCCAAGGACATCTTCAAGGGCCTGGACAGCCTGACCAACGTGGACCTGAGAGGCAACTCCTTCAAC
TGCGACTGCAAGCTGAAGTGGCTGGTGGAATGGCTGGGCCACACCAACGCCACCGTGGAAGATATC
TACTGCGAGGGCCCACCCGAGTACAAGAAGCGGAAGATCAACAGCCTGTCCAGCAAGGACTTCGAC
TGCATCATCACCGAGTTCGCCAAGAGCCAGGACCTGCCCTACCAGAGCCTGAGCATCGACACCTTC
AGCTACCTGAACGACGAGTACGTCGTGATCGCCCAGCCCTTTACCGGCAAGTGCATCTTCCTGGAA
TGGGACCACGTGGAAAAGACCTTCCGGAACTACGACAACATCACCGGCACCAGCACCGTCGTGTGC
AAGCCCATCGTGATCGAGACACAGCTGTACGTGATCGTGGCTCAGCTGTTCGGCGGCAGCCACATC
TACAAGCGGGACAGCTTCGCCAACAAGTTCATCAAGATCCAGGACATCGAGATCCTGAAGATCCGG
AAGCCCAACGACATCGAAACCTTTAAGATTGAGAACAACTGGTACTTCGTGGTGGCCGACAGCAGC
AAGGCCGGCTTTACCACCATCTATAAGTGGAACGGCAACGGCTTCTACAGCCACCAGTCCCTGCAC
GCCTGGTACAGAGACACCGACGTGGAATATCTGGAAATCGTGCGGACCCCCCAGACCCTGAGAACC
CCTCACCTGATCCTGAGCAGCTCCAGCCAGCGGCCCGTGATCTACCAGTGGAACAAGGCCACCCAG
CTGTTTACCAACCAGACCGACATCCCCAACATGGAAGATGTGTACGCCGTGAAGCACTTCAGCGTG
AAGGGCGACGTGTACATCTGCCTGACCCGGTTCATCGGCGACAGCAAAGTGATGAAGTGGGGCGGC
AGCAGCTTCCAGGATATCCAGCGGATGCCCAGCCGGGGCAGCATGGTGTTTCAGCCCCTGCAGATC
AACAACTACCAGTACGCCATCCTGGGCAGCGACTACAGCTTCACCCAGGTGTACAATTGGGACGCC
GAGAAGGCCAAGTTCGTGAAGTTCCAGGAACTGAACGTGCAGGCCCCCAGATCCTTTACCCACGTG
TCAATCAACAAGCGGAATTTCCTGTTCGCCAGCTCCTTTAAGGGCAACACCCAGATCTATAAGCAC
GTGATCGTGGACCTGAGCGCCTGA*
SEQ ID NO: 4-vector sequence (AAV2/9-CAG-{codon optimised}hLGI1-IRES-copGFP)

| | |
|---|---|
| tcgacccggg cggccgcttc gagcagacat gataagatac attgatgagt ttggacaaac | 60 |
| cacaactaga atgcagtgaa aaaaatgctt tatttgtgaa atttgtgatg ctattgcttt | 120 |
| atttgtaacc attataagct gcaataaaca agttaacaac aacaattgca ttcattttat | 180 |
| gtttcaggtt cagggggaga tgtgggaggt tttttaaagc aagtaaaacc tctacaaatg | 240 |
| tggtaaaatc gataaggatc ttcctagagc atggctacgt agataagtag catggcgggt | 300 |
| taatcattaa ctacaaggaa cccctagtga tggagttggc cactccctct ctgcgcgctc | 360 |
| gctcgctcac tgaggccggg cgaccaaagg tcgcccgacg cccgggcttt gcccgggcgg | 420 |
| cctcagtgag cgagcgagcg cgcagctgca ttaatgaatc ggccaacgcg cggggagagg | 480 |
| cggtttgcgt attgggcgct cttccgcttc ctcgctcact gactcgctgc gctcggtcgt | 540 |
| tcggctgcgg cgagcggtat cagctcactc aaaggcggta atacggttat ccacagaatc | 600 |
| aggggataac gcaggaaaga acatgtgagc aaaaggccag caaaaggcca ggaaccgtaa | 660 |
| aaaggccgcg ttgctggcgt ttttccatag gctccgcccc cctgacgagc atcacaaaaa | 720 |
| tcgacgctca agtcagaggt ggcgaaaccc gacaggacta taaagatacc aggcgtttcc | 780 |
| ccctggaagc tccctcgtgc gctctcctgt tccgaccctg ccgcttaccg gatacctgtc | 840 |
| cgcctttctc ccttcgggaa gcgtggcgct ttctcatagc tcacgctgta ggtatctcag | 900 |
| ttcggtgtag gtcgttcgct ccaagctggg ctgtgtgcac gaaccccccg ttcagcccga | 960 |
| ccgctgcgcc ttatccggta actatcgtct tgagtccaac ccggtaagac acgacttatc | 1020 |
| gccactggca gcagccactg gtaacaggat tagcagagcg aggtatgtag gcggtgctac | 1080 |
| agagttcttg aagtggtggc ctaactacgg ctacactaga agaacagtat ttggtatctg | 1140 |
| cgctctgctg aagccagtta ccttcggaaa aagagttggt agctcttgat ccggcaaaca | 1200 |
| aaccaccgct ggtagcggtg gtttttttgt ttgcaagcag cagattacgc gcagaaaaaa | 1260 |
| aggatctcaa gaagatcctt tgatcttttc tacggggtct gacgctcagt ggaacgaaaa | 1320 |
| ctcacgttaa gggattttgg tcatgagatt atcaaaaagg atcttcacct agatcctttt | 1380 |
| aaattaaaaa tgaagtttta aatcaatcta aagtatatat gagtaaactt ggtctgacag | 1440 |
| ttaccaatgc ttaatcagtg aggcacctat ctcagcgatc tgtctatttc gttcatccat | 1500 |
| agttgcctga ctccccgtcg tgtagataac tacgatacgg gagggcttac catctggccc | 1560 |
| cagtgctgca atgataccgc gagacccacg ctcaccggct ccagatttat cagcaataaa | 1620 |
| ccagccagcc ggaagggccg agcgcagaag tggtcctgca actttatccg cctccatcca | 1680 |
| gtctattaat tgttgccggg aagctagagt aagtagttcg ccagttaata gtttgcgcaa | 1740 |
| cgttgttgcc attgctacag gcatcgtggt gtcacgctcg tcgtttggta tggcttcatt | 1800 |
| cagctccggt tcccaacgat caaggcgagt tacatgatcc cccatgttgt gcaaaaaagc | 1860 |
| ggttagctcc ttcggtcctc cgatcgttgt cagaagtaag ttggccgcag tgttatcact | 1920 |
| catggttatg gcagcactgc ataattctct tactgtcatg ccatccgtaa gatgcttttc | 1980 |
| tgtgactggt gagtactcaa ccaagtcatt ctgagaatag tgtatgcggc gaccgagttg | 2040 |
| ctcttgcccg gcgtcaatac gggataatac cgcgccacat agcagaactt taaaagtgct | 2100 |
| catcattgga aaacgttctt cggggcgaaa actctcaagg atcttaccgc tgttgagatc | 2160 |
| cagttcgatg taacccactc gtgcacccaa ctgatcttca gcatctttta ctttcaccag | 2220 |
| cgtttctggg tgagcaaaaa caggaaggca aaatgccgca aaaaagggaa taagggcgac | 2280 |
| acggaaatgt tgaatactca tactcttcct ttttcaatat tattgaagca tttatcaggg | 2340 |
| ttattgtctc atgagcggat acatatttga atgtatttag aaaaataaac aaataggggt | 2400 |
| tccgcgcaca tttccccgaa aagtgccacc taaattgtaa gcgttaatat tttgttaaaa | 2460 |
| ttcgcgttaa atttttgtta aatcagctca ttttttaacc aataggccga aatcggcaaa | 2520 |
| atcccttata aatcaaaaga atagaccgag atagggttga gtgttgttcc agtttggaac | 2580 |
| aagagtccac tattaaagaa cgtggactcc aacgtcaaag ggcgaaaaac cgtctatcag | 2640 |
| ggcgatggcc cactacgtga accatcaccc taatcaagtt ttttggggtc gaggtgccgt | 2700 |
| aaagcactaa atcggaaccc taaagggagc ccccgattta gagcttgacg gggaaagccg | 2760 |
| gcgaacgtgg cgagaaagga agggaagaaa gcgaaaggag cgggcgctag ggcgctggca | 2820 |
| agtgtagcgg tcacgctgcg cgtaaccacc acacccgccg cgcttaatgc gccgctacag | 2880 |
| ggcgcgtccc attcgccatt caggctgcgc aactgttggg aagggcgatc ggtgcgggcc | 2940 |
| tcttcgctat tacgccagct gcgcgctcgc tcgctcactg aggccgcccg ggcaaagccc | 3000 |
| gggcgtcggg cgacctttgg tcgcccggcc tcagtgagcg agcgagcgcg cagagaggga | 3060 |
| gtggccaact ccatcactag gggttccttg tagttaatga ttaacccgcc atgctactta | 3120 |
| tctacgtagc catgctctag gaagagtacc attgacgtca ataatgacgt atgttcccat | 3180 |
| agtaacgcca atagggactt tccattgacg tcaatgggtg gagtatttac ggtaaactgc | 3240 |
| ccacttggca gtacatcaag tgtatcatat gccaagtacg ccccctattg acgtcaatga | 3300 |
| cggtaaatgg cccgcctggc attatgccca gtacatgacc ttatgggact ttcctacttg | 3360 |
| gcagtacatc tacgtattag tcatcgctat taccatggtc gaggtgagcc ccacgttctg | 3420 |
| cttcactctc cccatctccc ccccctcccc acccccaatt ttgtatttat ttatttttta | 3480 |
| attattttgt gcagcgatgg gggcgggggg gggggggggg cgcgcgccgg gcggggcggg | 3540 |
| gcggggcgag gggcggggcg gggcgaggcg gagaggtgcg gcggcagcca atcagagcgg | 3600 |
| cgcgctccga aagtttcctt ttatggcgag gcggcggcgg cggcggccct ataaaaagcg | 3660 |
| aagcgcgcgg cgggcgggag tcgctgcgcg ctgccttcgc cccgtgcccc gctccgccgc | 3720 |
| cgcctcgcgc cgcccgcccc ggctctgact gaccgcgtta ctcccacagg tgagcgggcg | 3780 |
| ggacggccct tctcctccgg gctgtaatta gcgcttggtt taatgacggc ttgtttcttt | 3840 |
| tctgtggctg cgtgaaagcc ttgaggggct ccgggagggc cctttgtgcg gggggagcgg | 3900 |
| ctcggggctg tccgcggggg gacggctgcc ttcggggggg acggggcagg gcggggttcg | 3960 |
| gcttctggcg tgtgaccggc ggctctagag cctctgctaa ccatgttcat gccttcttct | 4020 |
| ttttcctaca gctcctgggc aacgtgctgg ttattgtgct gtctcatcat tttggcaaag | 4080 |
| aattggatcc agctagcatg gaaagcgagc ggagcaagcg gatgggcaac gcctgcatcc | 4140 |
| ccctgaagcg gatcgcctac ttcctgtgcc tgctgagcgc cctgctgctg accgagggaa | 4200 |
| agaagcccgc caagcctaag tgccctgccg tgtgcacctg taccaaggac aacgccctgt | 4260 |
| gcgagaacgc ccggtccatc cctagaaccg tgccccccga tgtgatcagc ctgagctttg | 4320 |
| tgcggagcgg cttcaccgag atcagcgagg gcagcttcct gttcaccccc agcctgcagc | 4380 |
| tgctgctgtt caccagcaac agcttcgacg tgatcagcga cgacgccttc atcggcctgc | 4440 |
| cccacctgga atacctgttc atcgagaaca acaacatcaa gagcatcagc cggcacacct | 4500 |
| tccggggcct gaagtccctg atccacctga gcctggccaa caacaacctg cagaccctgc | 4560 |
| ccaaggacat cttcaagggc ctggacagcc tgaccaacgt ggacctgaga ggcaactcct | 4620 |
| tcaactgcga ctgcaagctg aagtggctgg tggaatggct gggccacacc aacgccaccg | 4680 |
| tggaagatat ctactgcgag ggcccacccg agtacaagaa gcggaagatc aacagcctgt | 4740 |
| ccagcaagga cttcgactgc atcatcaccg agttcgccaa gagccaggac ctgccctacc | 4800 |
| agagcctgag catcgacacc ttcagctacc tgaacgacga gtacgtcgtg atcgcccagc | 4860 |
| cctttaccgg caagtgcatc ttcctggaat gggaccacgt ggaaaagacc ttccggaact | 4920 |
| acgacaacat caccggcacc agcaccgtcg tgtgcaagcc catcgtgatc gagacacagc | 4980 |
| tgtacgtgat cgtggctcag ctgttcggcg gcagccacat ctacaagcgg gacagcttcg | 5040 |
| ccaacaagtt catcaagatc caggacatcg agatcctgaa gatccggaag cccaacgaca | 5100 |
| tcgaaacctt taagattgag aacaactggt acttcgtggt ggccgacagc agcaaggccg | 5160 |
| gctttaccac catctataag tggaacggca acggcttcta cagccaccag tccctgcacg | 5220 |
| cctggtacag agacaccgac gtggaatatc tggaaatcgt gcggaccccc cagaccctga | 5280 |
| gaacccctca cctgatcctg agcagctcca gccagcggcc cgtgatctac cagtggaaca | 5340 |
| aggccaccca gctgtttacc aaccagaccg acatccccaa catggaagat gtgtacgccg | 5400 |
| tgaagcactt cagcgtgaag ggcgacgtgt acatctgcct gacccggttc atcggcgaca | 5460 |
| gcaaagtgat gaagtggggc ggcagcagct tccaggatat ccagcggatg cccagccggg | 5520 |
| gcagcatggt gtttcagccc ctgcagatca acaactacca gtacgccatc ctgggcagcg | 5580 |
| actacagctt cacccaggtg tacaattggg acgccgagaa ggccaagttc gtgaagttcc | 5640 |
| aggaactgaa cgtgcaggcc cccagatcct ttacccacgt gtcaatcaac aagcggaatt | 5700 |
| tcctgttcgc cagctccttt aagggcaaca cccagatcta taagcacgtg atcgtggacc | 5760 |
| tgagcgcctg aacccagctt tcttgtacaa agtgggcccc tctccctccc ccccccctaa | 5820 |
| cgttactggc cgaagccgct tggaataagg ccggtgtgcg tttgtctata tgttattttc | 5880 |
| caccatattg ccgtcttttg gcaatgtgag ggcccggaaa cctggccctg tcttcttgac | 5940 |
| gagcattcct aggggtcttt cccctctcgc caaaggaatg caaggtctgt tgaatgtcgt | 6000 |
| gaaggaagca gttcctctgg aagcttcttg aagacaaaca acgtctgtag cgaccctttg | 6060 |
| caggcagcgg aaccccccac ctggcgacag gtgcctctgc ggccaaaagc cacgtgtata | 6120 |
| agatacacct gcaaaggcgg cacaacccca gtgccacgtt gtgagttgga tagttgtgga | 6180 |
| aagagtcaaa tggctctcct caagcgtatt caacaagggg ctgaaggatg cccagaaggt | 6240 |
| accccattgt atgggatctg atctggggcc tcggtgcaca tgctttacat gtgtttagtc | 6300 |
| gaggttaaaa aaacgtctag gccccccgaa ccacggggac gtggttttcc tttgaaaaac | 6360 |
| acgatgataa tatggccaca accatgcccg ccatgaagat cgagtgccgc atcaccggca | 6420 |
| ccctgaacgg cgtggagttc gagctggtgg gcggcggaga gggcaccccc gagcagggcc | 6480 |
| gcatgaccaa caagatgaag agcaccaaag gcgccctgac cttcagcccc tacctgctga | 6540 |
| gccacgtgat gggctacggc ttctaccact tcggcaccta ccccagcggc tacgagaacc | 6600 |
| ccttcctgca cgccatcaac aacggcggct acaccaacac ccgcatcgag aagtacgagg | 6660 |
| acggggcgt gctgcacgtg agcttcagct accgctacga ggccggccgc gtgatcggcg | 6720 |
| acttcaaggt ggtgggcacc ggcttccccg aggacagcgt gatcttcacc gacaagatca | 6780 |
| tccgcagcaa cgccaccgtg gagcacctgc accccatggg cgataacgtg ctggtgggca | 6840 |
| gcttcgcccg caccttcagc ctgcgcgacg gcggctacta cagcttcgtg gtggacagcc | 6900 |
| acatgcactt caagagcgcc atccacccca gcatcctgca gaacgggggc cccatgttcg | 6960 |
| ccttccgccg cgtggaggag ctgcacagca acaccgagct gggcatcgtg gagtaccagc | 7020 |
| acgccttcaa gacccccatc gccttcgcca gatctcgaga tatcagccat ggcttcccgc | 7080 |
| cggcggtggc ggcgcaggat gatggcacgc tgcccatgtc ttgtgcccag gagagcggga | 7140 |
| tggaccgtca ccctgcagcc tgtgcttctg ctaggatcaa tgtgaccggt taacg* | 7195 |

SEQ ID NO: 5-vector sequence (AAV2/9-hSyn-{codon optimised}hLGI1-IRES2-eGFP)

| | |
|---|---|
| cctgcaggca gctgcgcgct cgctcgctca ctgaggccgc ccgggcaaag cccgggcgtc | 60 |
| gggcgacctt tggtcgcccg gcctcagtga gcgagcgagc gcgcagagag ggagtggcca | 120 |
| actccatcac taggggttcc tgcggccgca cgcgtgaatt cgagtgcaag tgggttttag | 180 |
| gaccaggatg aggcggggtg ggggtgccta cctgacgacc gaccccaccc actggacaag | 240 |
| cacccaaccc ccattcccca aattgcgcat cccctatcag agagggggag gggaaacagg | 300 |
| atgcggcgag gcgcgtgcgc actgccagct tcagcaccgc ggacagtgcc ttcgcccccg | 360 |
| cctggcggcg cgcgccaccg ccgcctcagc actgaaggcg cgctgacgtc actcgccggt | 420 |
| cccccgcaaa ctccccttcc cggccacctt ggtcgcgtcc gcgccgccgc cggcccagcc | 480 |
| ggaccgcacc acgcgaggcg cgagataggg gggcacgggc gcgaccatct gcgctgcggc | 540 |
| gccggcgact cagcgctgcc tcagtctgcg gtgggcagcg gaggagtcgt gtcgtgcctg | 600 |
| agagcgcagt cgaattggat ccAGCTAGCA TGGAAAGCGA GCGGAGCAAG CGGATGGGCA | 660 |
| ACGCCTGCAT CCCCCTGAAG CGGATCGCCT ACTTCCTGTG CCTGCTGAGC GCCCTGCTGC | 720 |
| TGACCGAGGG AAAGAAGCCC GCCAAGCCTA AGTGCCCTGC CGTGTGCACC TGTACCAAGG | 780 |
| ACAACGCCCT GTGCGAGAAC GCCCGGTCCA TCCCTAGAAC CGTGCCCCCC GATGTGATCA | 840 |
| GCCTGAGCTT TGTGCGGAGC GGCTTCACCG AGATCAGCGA GGGCAGCTTC CTGTTCACCC | 900 |
| CCAGCCTGCA GCTGCTGCTG TTCACCAGCA ACAGCTTCGA CGTGATCAGC GACGACGCCT | 960 |
| TCATCGGCCT GCCCCACCTG GAATACCTGT TCATCGAGAA CAACAACATC AAGAGCATCA | 1020 |
| GCCGGCACAC CTTCCGGGGC CTGAAGTCCC TGATCCACCT GAGCCTGGCC AACAACAACC | 1080 |
| TGCAGACCCT GCCCAAGGAC ATCTTCAAGG GCCTGGACAG CCTGACCAAC GTGGACCTGA | 1140 |
| GAGGCAACTC CTTCAACTGC GACTGCAAGC TGAAGTGGCT GGTGGAATGG CTGGGCCACA | 1200 |
| CCAACGCCAC CGTGGAAGAT ATCTACTGCG AGGGCCCACC CGAGTACAAG AAGCGGAAGA | 1260 |
| TCAACAGCCT GTCCAGCAAG GACTTCGACT GCATCATCAC CGAGTTCGCC AAGAGCCAGG | 1320 |
| ACCTGCCCTA CCAGAGCCTG AGCATCGACA CCTTCAGCTA CCTGAACGAC GAGTACGTCG | 1380 |
| TGATCGCCCA GCCCTTTACC GGCAAGTGCA TCTTCCTGGA ATGGGACCAC GTGGAAAAGA | 1440 |
| CCTTCCGGAA CTACGACAAC ATCACCGGCA CCAGCACCGT CGTGTGCAAG CCCATCGTGA | 1500 |
| TCGAGACACA GCTGTACGTG ATCGTGGCTC AGCTGTTCGG CGGCAGCCAC ATCTACAAGC | 1560 |
| GGGACAGCTT CGCCAACAAG TTCATCAAGA TCCAGGACAT CGAGATCCTG AAGATCCGGA | 1620 |
| AGCCCAACGA CATCGAAACC TTTAAGATTG AGAACAACTG GTACTTCGTG GTGGCCGACA | 1680 |
| GCAGCAAGGC CGGCTTTACC ACCATCTATA AGTGGAACGG CAACGGCTTC TACAGCCACC | 1740 |
| AGTCCCTGCA CGCCTGGTAC AGAGACACCG ACGTGGAATA TCTGGAAATC GTGCGGACCC | 1800 |
| CCCAGACCCT GAGAACCCCT CACCTGATCC TGAGCAGCTC CAGCCAGCGG CCCGTGATCT | 1860 |
| ACCAGTGGAA CAAGGCCACC CAGCTGTTTA CCAACCAGAC CGACATCCCC AACATGGAAG | 1920 |
| ATGTGTACGC CGTGAAGCAC TTCAGCGTGA AGGGCGACGT GTACATCTGC CTGACCCGGT | 1980 |
| TCATCGGCGA CAGCAAAGTG ATGAAGTGGG GCGGCAGCAG CTTCCAGGAT ATCCAGCGGA | 2040 |
| TGCCCAGCCG GGGCAGCATG GTGTTTCAGC CCCTGCAGAT CAACAACTAC CAGTACGCCA | 2100 |
| TCCTGGGCAG CGACTACAGC TTCACCCAGG TGTACAATTG GGACGCCGAG AAGGCCAAGT | 2160 |
| TCGTGAAGTT CCAGGAACTG AACGTGCAGG CCCCCAGATC CTTTACCCAC GTGTCAATCA | 2220 |
| ACAAGCGGAA TTTCCTGTTC GCCAGCTCCT TTAAGGGCAA CACCCAGATC TATAAGCACG | 2280 |
| TGATCGTGGA CCTGAGCGCC TGAACCCAGC TTTCTTGTAC AAAGTGGGCC CCTCTCCCTC | 2340 |
| CCCCCCCCCT AACGTTACTG GCCGAAGCCG CTTGGAATAA GGCCGGTGTG CGTTTGTCTA | 2400 |
| TATGTTATTT TCCACCATAT TGCCGTCTTT TGGCAATGTG AGGGCCCGGA AACCTGGCCC | 2460 |
| TGTCTTCTTG ACGAGCATTC CTAGGGGTCT TTCCCCTCTC GCCAAAGGAA TGCAAGGTCT | 2520 |
| GTTGAATGTC GTGAAGGAAG CAGTTCCTCT GGAAGCTTCT TGAAGACAAA CAACGTCTGT | 2580 |
| AGCGACCCTT TGCAGGCAGC GGAACCCCCC ACCTGGCGAC AGGTGCCTCT GCGGCCAAAA | 2640 |
| GCCACGTGTA TAAGATACAC CTGCAAAGGC GGCACAACCC CAGTGCCACG TTGTGAGTTG | 2700 |
| GATAGTTGTG GAAAGAGTCA AATGGCTCTC CTCAAGCGTA TTCAACAAGG GGCTGAAGGA | 2760 |
| TGCCCAGAAG GTACcccatt gtatgggatc tgatctgggg cctcggtgca catgctttac | 2820 |
| atgtgtttag tcgaggttaa aaaaacgtct aggccccccg aaccacgggg acgtggtttt | 2880 |
| cctttgaaaa acacgatgat aatatggcca caaccatggt gagcaagggc gaggagctgt | 2940 |
| tcaccggggt ggtgcccatc ctggtcgagc tggacggcga cgtaaacggc cacaagttca | 3000 |
| gcgtgtccgg cgagggcgag ggcgatgcca cctacggcaa gctgaccctg aagttcatct | 3060 |
| gcaccaccgg caagctgccc gtgccctggc ccaccctcgt gaccaccctg acctacggcg | 3120 |
| tgcagtgctt cagccgctac cccgaccaca tgaagcagca cgacttcttc aagtccgcca | 3180 |
| tgcccgaagg ctacgtccag gagcgcacca tcttcttcaa ggacgacggc aactacaaga | 3240 |
| cccgcgccga ggtgaagttc gagggcgaca ccctggtgaa ccgcatcgag ctgaagggca | 3300 |
| tcgacttcaa ggaggacggc aacatcctgg ggcacaagct ggagtacaac tacaacagcc | 3360 |
| acaacgtcta tatcatggcc gacaagcaga agaacggcat caaggtgaac ttcaagatcc | 3420 |
| gccacaacat cgaggacggc agcgtgcagc tcgccgacca ctaccagcag aacaccccca | 3480 |
| tcggcgacgg ccccgtgctg ctgcccgaca accactacct gagcacccag tccgccctga | 3540 |
| gcaaagaccc caacgagaag cgcgatcaca tggtcctgct ggagttcgtg accgccgccg | 3600 |
| ggatcactct cggcatggac gagctgtaca agtaaGtcga caatcaacct ctggattaca | 3660 |
| aaatttgtga aagattgact ggtattctta actatgttgc tccttttacg ctatgtggat | 3720 |
| acgctgcttt aatgcctttg tatcatgcta ttgcttcccg tatggctttc attttctcct | 3780 |
| ccttgtataa atcctggttg ctgtctcttt atgaggagtt gtggcccgtt gtcaggcaac | 3840 |
| gtggcgtggt gtgcactgtg tttgctgacg caacccccac tggttggggc attgccacca | 3900 |
| cctgtcagct cctttccggg actttcgctt tccccctccc tattgccacg gcggaactca | 3960 |
| tcgccgcctg ccttgcccgc tgctggacag gggctcggct gttgggcact gacaattccg | 4020 |
| tggtgttgtc ggggaaatca tcgtcctttc cttggctgct cgcctgtgtt gccacctgga | 4080 |
| ttctgcgcgg gacgtccttc tgctacgtcc cttcggccct caatccagcg gaccttcctt | 4140 |
| cccgcggcct gctgccggct ctgcggcctc ttccgcgtct tcgccttcgc cctcagacga | 4200 |
| gtcggatctc cctttgggcc gcctccccgc atcgataccg agcgctgctc gagagatcta | 4260 |
| cgggtggcat ccctgtgacc cctccccagt gcctctcctg gccctggaag ttgccactcc | 4320 |
| agtgcccacc agccttgtcc taataaaatt aagttgcatc attttgtctg actaggtgtc | 4380 |
| cttctataat attatggggt ggaggggggt ggtatggagc aaggggcaag ttgggaagac | 4440 |
| aacctgtagg gcctgcgggg tctattggga accaagctgg agtgcagtgg cacaatcttg | 4500 |
| gctcactgca atctccgcct cctgggttca agcgattctc ctgcctcagc ctcccgagtt | 4560 |
| gttgggattc caggcatgca tgaccaggct cagctaattt ttgttttttt ggtagagacg | 4620 |
| gggtttcacc atattggcca ggctggtctc caactcctaa tctcaggtga tctacccacc | 4680 |
| ttggcctccc aaattgctgg gattacaggc gtgaaccact gctcccttcc ctgtccttct | 4740 |
| gattttgtag gtaaccacgt gcggaccgag cggccgcagg aacccctagt gatggagttg | 4800 |
| gccactccct ctctgcgcgc tcgctcgctc actgaggccg ggcgaccaaa ggtcgcccga | 4860 |
| cgcccgggct ttgcccgggc ggcctcagtg agcgagcgag cgcgcagctg cctgcagggg | 4920 |
| cgcctgatgc ggtattttct ccttacgcat ctgtgcggta tttcacaccg catacgtcaa | 4980 |
| agcaaccata gtacgcgccc tgtagcggcg cattaagcgc ggcgggtgtg gtggttacgc | 5040 |
| gcagcgtgac cgctacactt gccagcgccc tagcgcccgc tcctttcgct ttcttccctt | 5100 |
| cctttctcgc cacgttcgcc ggctttcccc gtcaagctct aaatcggggg ctccctttag | 5160 |
| ggttccgatt tagtgcttta cggcacctcg accccaaaaa acttgattag ggtgatggtt | 5220 |
| cacgtagtgg gccatcgccc tgatagacgg tttttcgccc tttgacgttg gagtccacgt | 5280 |
| tctttaatag tggactcttg ttccaaactg gaacaacact caaccctatc tcggtctatt | 5340 |
| cttttgattt ataagggatt ttgccgattt cggcctattg gttaaaaaat gagctgattt | 5400 |
| aacaaaaatt taacgcgaat tttaacaaaa tattaacgct tacaatttag gtggcacttt | 5460 |
| tcggggaaat gtgcgcggaa cccctatttg tttatttttc taaatacatt caaatatgta | 5520 |
| tccgctcatg agacaataac cctgataaat gcttcaataa tagcacctag atcaagagac | 5580 |
| aggatgagga tcgtttcgca tgattgaaca agatggattg cacgcaggtt ctccggccgc | 5640 |
| ttgggtggag aggctattcg gctatgactg ggcacaacag acaatcggct gctctgatgc | 5700 |
| cgccgtgttc cggctgtcag cgcaggggcg cccggttctt tttgtcaaga ccgacctgtc | 5760 |
| cggtgccctg aatgaactgc aagacgaggc agcgcggcta tcgtggctgg ccacgacggg | 5820 |
| cgttccttgc gcagctgtgc tcgacgttgt cactgaagcg ggaagggact ggctgctatt | 5880 |
| gggcgaagtg ccggggcagg atctcctgtc atctcacctt gctcctgccg agaaagtatc | 5940 |
| catcatggct gatgcaatgc ggcggctgca tacgcttgat ccggctacct gcccattcga | 6000 |
| ccaccaagcg aaacatcgca tcgagcgagc acgtactcgg atggaagccg gtcttgtcga | 6060 |
| tcaggatgat ctggacgaag agcatcaggg gctcgcgcca gccgaactgt tcgccaggct | 6120 |
| caaggcgagc atgcccgacg gcgaggatct cgtcgtgacc catggcgatg cctgcttgcc | 6180 |
| gaatatcatg gtggaaaatg gccgcttttc tggattcatc gactgtggcc ggctgggtgt | 6240 |
| ggcggaccgc tatcaggaca tagcgttggc tacccgtgat attgctgaag agcttggcgg | 6300 |
| cgaatgggct gaccgcttcc tcgtgcttta cggtatcgcc gctcccgatt cgcagcgcat | 6360 |
| cgccttctat cgccttcttg acgagttctt ctgaattatt aacgcttaca atttcctgat | 6420 |
| gcggtatttt ctccttacgc atctgtgcgg tatttcacac cgcatcaggt ggcacttttc | 6480 |
| ggggaaatgt gcgcggaacc cctatttgtt tatttttcta aatacattca aatatgtatc | 6540 |
| cgctcatgac caaaatccct taacgtgagt tttcgttcca ctgagcgtca gaccccgtag | 6600 |
| aaaagatcaa aggatcttct tgagatcctt tttttctgcg cgtaatctgc tgcttgcaaa | 6660 |
| caaaaaaacc accgctacca gcggtggttt gtttgccgga tcaagagcta ccaactcttt | 6720 |
| ttccgaaggt aactggcttc agcagagcgc agataccaaa tactgttctt ctagtgtagc | 6780 |
| cgtagttagg ccaccacttc aagaactctg tagcaccgcc tacatacctc gctctgctaa | 6840 |
| tcctgttacc agtggctgct gccagtggcg ataagtcgtg tcttaccggg ttggactcaa | 6900 |
| gacgatagtt accggataag gcgcagcggt cgggctgaac gggggggtcg tgcacacagc | 6960 |
| ccagcttgga gcgaacgacc tacaccgaac tgagatacct acagcgtgag ctatgagaaa | 7020 |
| gcgccacgct tcccgaaggg agaaaggcgg acaggtatcc ggtaagcggc agggtcggaa | 7080 |
| caggagagcg cacgagggag cttccagggg gaaacgcctg gtatctttat agtcctgtcg | 7140 |
| ggtttcgcca cctctgactt gagcgtcgat ttttgtgatg ctcgtcaggg gggcggagcc | 7200 |
| tatggaaaaa cgccagcaac gcggcctttt tacggttcct ggccttttgc tggccttttg | 7260 |
| ctcacatgt | 7269 |

1. A method of treatment of acquired focal epilepsy in a human subject in need of the same, the method comprising:
(i) providing an expression vector encoding a polynucleotide sequence encoding LGI1, wherein the polynucleotide sequence is operably linked to a promoter suitable to drive expression of the LGI1 in human cells,
(ii) administering the expression vector to the subject.

2. The method as claimed in claim 1 wherein the polynucleotide sequence encoding LGI1 encodes an amino acid sequence comprising or consisting the amino acid sequence shown in SEQ ID NO: 2 or a homologous variant thereof.

3. The method as claimed in claim 2 wherein the polynucleotide sequence encoding LGI1 has a nucleotide sequence comprising or consisting of the nucleotide sequence shown in SEQ ID NO: 1 or a homologous variant thereof, which is optionally a codon optimised sequence, which is optionally shown in SEQ ID NO: 3.

4. The method as claimed in any one of claims 1 to 3 wherein the promoter is a cell type specific promoter.

5. The method as claimed in claim 4 wherein the cell type specific promoter is specific for neurons.

6. The method as claimed in any one of claims 1 to 5 wherein the promoter is a CAG promoter or human synapsin promoter.

7. The method as claimed in any one of claims 1 to 6 wherein the vector is a viral vector.

8. The method as claimed in claim 7 wherein the vector is an AAV vector.

9. The method as claimed in claim 8 wherein the AAV vector is an AAV9 vector, optionally an AAV2/9 vector.

10. The method as claimed in claim 7 or claim 8 wherein the vector has the nucleotide sequence of SEQ ID NO: 4 or a homologous variant thereof, optionally lacking the GFP sequence.

11. The method as claimed in any one of claims 7 to 10 which comprises the steps of:
(i) assembling viral particles in vitro by transducing mammalian cells with the expression vector and expressing viral packaging and envelope proteins necessary for particle formation in the cells and culturing the transduced cells in a culture medium, such that the cells produce viral particles comprising the expression vector that are released into the medium;
(ii) administering the viral particles to the subject.

12. The method of claim 11, wherein the method comprises transducing the mammalian cells with one or more viral packaging and envelope expression vectors that encode the viral packaging and envelope proteins necessary for particle formation.

13. The method as claimed in any one of claims 1 to 12 wherein the expression vector or viral particles comprising the expression vector are administered directly to a CNS site in the subject.

14. The method as claimed in any one of claims 1 to 13 wherein the acquired focal epilepsy affects a single area of the brain.

15. The method as claimed in any one of claims 1 to 13 wherein the acquired focal epilepsy affects multiple discrete areas of the brain.

16. The method as claimed in any one of claims 1 to 15 wherein the acquired focal epilepsy is temporal lobe epilepsy with hippocampal sclerosis.

17. An expression vector or viral particle comprising an expression vector for use in a method of treatment of acquired focal epilepsy in a human subject as claimed in any one of claims 1 to 16.

18. A method of treatment of acquired focal epilepsy in a human subject comprising use of an expression vector or viral particle comprising an expression vector as defined in any one of claims 1 to 16.

19. Use of an expression vector or use of a viral particle comprising an expression vector as defined in any one of claims 1 to 16 in the manufacture of a medicament for the treatment of acquired focal epilepsy in a human subject.

20. Use of an expression vector or use of a viral particle comprising an expression vector in the manufacture of a medicament for the treatment of acquired focal epilepsy in a human subject, wherein the treatment is as claimed in any one of claims 1 to 16.
